(19) United States
(12) Patent Application Publication
(10) Pub. No.: US 2015/0196452 A1
(43) Pub. Date: Jul. 16, 2015

(54) PARTICLE CONTAINERS AND DELIVERY APPLICATORS

(71) Applicant: Sebacia, Inc., Duluth, GA (US)

(72) Inventors: Todd J. Meyer, Rosewell, GA (US); Dilip Paithankar, Wayland, MA (US); Arthur Stone Rousmaniere, Hollis, NH (US); Derek John Hugger, Goffstown, NH (US); Joseph Omer St. Cyr., Salem, NH (US); John William Cain, Elk River, MN (US)

(21) Appl. No.: 14/593,992

(22) Filed: Jan. 9, 2015

Related U.S. Application Data
(60) Provisional application No. 61/926,211, filed on Jan. 10, 2014.

(51) Int. Cl. A61H 9/00; A61M 35/00

(52) U.S. Cl. CPC A61H 9/0021; A61M 35/00; A61H 2201/105

(57) ABSTRACT

Certain therapies involving delivering materials, such as light absorbing particles, to the surface of a subject's skin. Thereafter, the movement of the material into skin structures, such as follicles, is facilitated by a mechanical device such as a massager. The present application describes certain devices useful in such therapies.

### CROSS REFERENCE TO RELATED APPLICATION

This application claims the benefit or priority under 35 U.S.C. §119(e) of U.S. Provisional Application Nos. 61/926,211 filed Jan. 10, 2014, the entire contents of which are incorporated herein by this reference.

### INCORPORATION BY REFERENCE

All publications and patent applications mentioned in this specification are herein incorporated by reference in their entirety to the same extent as if each individual publication or patent application was specifically and individually indicated to be incorporated by reference.

### FIELD

This application relates to containers suited for the storage of particle formulations. Also included are various related delivery systems suited to particle delivery alone or in integrated configurations with the particle storage container.

### BACKGROUND

Numerous selective photothermolysis treatments based on the external application to the body of light absorbing material have been proposed. One exemplary treatment includes the introduction of light absorbing material into the sebaceous follicle followed by laser irradiation as a treatment of acne. An example of the light absorbing material used in such procedures are certain nanoshells such as those having a silica core and a gold shell that were developed by Halas and others at Rice University. These materials can be expensive, difficult to work with and it is desired to reduce the amount used. Typically, a treatment involves application of a suspension of the particles to the skin surface followed by mechanical methods such as a massage. These particles are suspended in a liquid that typically consists of water, ethanol, diisopropyl adipate, and polyethyelene glycol. These are typically low viscosity formulations and particle delivery is aided by a number of different mechanical means.

The increased use of such systems coupled with the challenges of providing formulations to various skin treatment locations has revealed shortcomings in existing particle delivery and delivery assistance systems. What is needed are improved particle formulation storage containers as stand-alone components, or optionally, as integral components to a particle delivery system utilized to facilitate delivery of particles to desired skin target sites.

### SUMMARY OF THE DISCLOSURE

The present invention relates to various alternative configurations of particle containers, both disposable and those integrated into and re-useable with the applicator.

There are also a number of alternative particle penetration assistance mechanisms. While illustrated as separate from a particle delivery configuration in some embodiments, the particle penetration assistance mechanisms and devices may be modified to cooperate with a particle formulation container or other particle formulation delivery technique described herein.

### BRIEF DESCRIPTION OF THE DRAWINGS

The novel features of the invention are set forth with particularity in the claims that follow. A better understanding of the features and advantages of the present invention will be obtained by reference to the following detailed description that sets forth illustrative embodiments, in which the principles of the invention are utilized, and the accompanying drawings of which:

FIGS. 1A-1F illustrate various views of a separate particle fluid dispenser pack and use with an exemplary applicator (FIG. 1F).

FIGS. 2A and 2B illustrate isometric and in use views, respectively, of an applicator pen. FIG. 2C is an exploded view and FIG. 2D is an alternative configuration.

FIGS. 3A, 3B and 3C illustrate in use, side and cross section views, respectively, of a roller ball applicator. FIGS. 3D and 3E provide alternative views.

FIG. 4A illustrates an in use, isometric view of a fingertip dispenser and hand held applicator.

FIG. 4B is a cross section view of an alternative fingertip dispenser embodiment.

FIGS. 4C-4F are additional views of alternative fingertip dispenser embodiments.

FIG. 5A illustrates an in use, isometric view of a disposable delivery container integrated into a hand held applicator.

FIG. 5B is an isometric view of the treatment head before attachment to the applicator and attached in FIG. 5D.

FIG. 5C is the particle container before attachment to the applicator as shown in FIG. 5A.

FIG. 6A illustrates an in use, isometric view of a disposable delivery container integrated into a hand held applicator.

FIG. 6B is an isometric view of the applicator before insertion of the particle container.

FIG. 6C is an isometric view of the particle container shown in FIG. 6A.

FIG. 7A illustrates an in use, isometric view of a disposable delivery container integrated into a hand held applicator.

FIG. 7B is a section view of the application in FIG. 7A illustrating the rack and pinion drive mechanism with a disposable delivery container integrated into a hand held applicator.

FIG. 7C illustrates a rear view of the applicator of FIG. 7A.

FIG. 7 D is an isometric view of the particle container shown in FIG. 7B.

FIG. 7 E illustrates an isometric view of an embodiment of a disposable delivery container integrated into a hand held applicator/massage device.

FIG. 7 F illustrates an isometric view of the embodiment of FIG. 7 E.

FIG. 7 G illustrates an isometric exploded view of the embodiment of FIG. 7 E in which the head cover is removed from the device.

FIG. 7 H illustrates a further isometric exploded view of the embodiment of FIG. 7 E in which the disposable delivery container is removed from the device.

FIG. 7 I illustrates an isometric view of a disposable delivery container, a filling tip, and a plunger.

FIG. 7 J illustrates an isometric view of a disposable delivery container, a filling tip engaged with said disposable delivery container, and a plunger in a partially retracted position as well as a filling vial.

FIG. 7 K illustrates an isometric view of a disposable delivery container, a delivery tip engaged with said disposable delivery container, and a stopper in a fully retracted position, but without the removable plunger.

FIG. 8A illustrates an isometric view of a disposable delivery container prior to insertion into a hand held applicator.

FIG. 8B is a section view of the applicator and inserted container of FIG. 8B.

FIG. 9 illustrates an isometric view of a dual ended hand held applicator.

FIG. 10A illustrates an enlarged view of the dispensing end of a dual ended applicator with a particle container in place.

FIG. 10B illustrates the insertion of a particle container into the dispensing end of a dual ended applicator.

FIG. 10C is an isometric view of the particle container illustrated in FIGS. 10A and 10B.

FIGS. 11A-11D illustrate various views of an adjustable massager head.

FIG. 12A illustrates an isometric view of a hand held applicator.

FIG. 12B is a view of the applicator of FIG. 12A with the head removed.

FIG. 13A illustrates an isometric view of a hand held applicator.

FIG. 13B is a view of the applicator of FIG. 13A with the head removed.

FIGS. 14A-14C illustrate various views of an palm grip dispenser.

FIG. 15A illustrates a palm dispenser of FIG. 14A-14C in use with an external battery and motor pack.

FIG. 15B illustrates the removal of the battery back.

FIG. 16 is an isometric view of a delivery container having a collapsible bag within a rigid casing.

FIG. 17 is an isometric view of a bulb pump and syringe delivery container having a collapsible bag within a syringe body.

FIG. 18 is an isometric view of a syringe attached to a delivery container having a collapsible squeeze reservoir and a pair of check valves.

FIG. 19 is an isometric view of a syringe attached to a delivery container having a collapsible squeeze reservoir and a pair of check valves.

FIG. 20 is an isometric view of a syringe attached to a delivery container having a collapsible squeeze reservoir and a pair of check valves.

FIG. 21 is an isometric view of a syringe attached to a delivery container having a collapsible squeeze reservoir and a single check valve.

FIGS. 22A and 22B illustrate a syringe attached to a compressible bellows delivery reservoir and a pair of check valves. The bellows are shown in an expanded configuration (FIG. 22A) and compressed configuration (FIG. 22B).

FIG. 23 is a table comparing the embodiments of FIGS. 18, 19, 20, 22A and 22B.

FIG. 24 is a cross section view of a piston pump fluid delivery device.

FIG. 25A is a comparison of the overall length of the various delivery devices of FIGS. 18, 24, 22A and 22B.

FIGS. 25B and 25C illustrate isometric and cross section views of a particle delivery housing.

FIG. 26 is an isometric view of a syringe body with a zip tie drive system.

FIG. 27 is an isometric view of a syringe body with a plunger drive device.

FIGS. 28A and 28B are isometric and section views of push rod delivery devices.

FIGS. 29A and 29B illustrate, respectively, the delivery devices of FIGS. 24 and 28A within an exemplary hand held applicator.

FIGS. 29C and 29D are side and rear isometric views, respectively of a hand held particle applicator.

FIG. 29E illustrates a prototype of the applicator in FIGS. 29C and 29D showing hand hold position and orientation to treatment surface in use.

FIGS. 29F and 29G are side and isometric views, respectively of a hand held particle applicator.

FIG. 29H illustrates a section view of the applicator of FIGS. 29F, 29G.

FIG. 29I is an enlarged view of the plunger actuator illustrated in the section view of FIG. 29H.

FIG. 30 is an isometric view of a fluid dispenser with a squeeze pad.

FIG. 31 is an enlarged view of the distal portion of a fluid dispenser having a material dispenser tip.

FIG. 32 is a bottom up isometric view of a single spring loaded roller ball fluid dispenser.

FIG. 33 is a bottom up isometric view of a multiple spring loaded roller ball fluid dispenser.

FIG. 34 is a bottom up isometric view of a multiple finger fluid dispenser.

FIG. 35A is a section view of a push to dispense fluid dispenser.

FIG. 35B is a section view of the dispenser of FIG. 35A illustrating how the delivery pores open when the tip is depressed.

FIG. 36 is an isometric view of a disposable sleeve on an applicator and particle containing dipping tray.

FIG. 37A is an isometric view of a double ended applicator.

FIG. 37B is an enlarged view of the fluid dispenser tip of the device in FIG. 37A in use with a dip applicator.

FIGS. 38A and 38B illustrate a cross section view of a push to dispense fluid delivery and applicator in the closed and fluid release configurations respectively.

FIG. 39A is a cross section view of spinning fluid dispenser and massage roller.

FIG. 39B is an isometric partial section view of the device of FIG. 39A.

FIG. 39C is a top down view of the device of FIG. 39A illustrating the rotating motion.

FIGS. 40A and 40B are isometric and cross section views of a fluid dispenser.

FIGS. 41A and 41B illustrate, respectively, a disposable dispensing cap before and after attachment to a mating receiver in an applicator.

FIG. 42A illustrates a section view of an applicator and foil packet particle packet prior to use.

FIG. 42B is an isometric view of the foil packet of FIG. 42A.

FIG. 43A illustrates a section view of an applicator and particle pad in use.

FIG. 43B is a top view of the pad in FIG. 43A.

FIG. 44A is an isometric view with exposed interior components of an applicator.

FIG. 44B is an enlarged view of an alternative configuration of the distal end of the applicator in FIG. 44A.

FIG. 45 is an isometric view of the distal end of an applicator having one or more elements to detect skin contact.

FIGS. 46A and 46B illustrate, respectively, a side view of an applicator and disposable dispenser separated and attached.

FIG. 46C is a top down view of a spinning fluid dispenser that interacts with the raised ear of the disposable dispenser.

FIG. 47 is an isometric view of a dispenser having a massager head and a wicking fluid delivery loop.

FIG. 48 is an enlarged isometric view of the distal end of a vibrating delivery head having a fluid tip and air nozzle delivery system.

FIG. 49 is an exposed side view of a spray done fluid delivery device.

FIGS. 50A and 50B illustrate isometric and side views, respectively, a fluid dispenser.

FIG. 51 is a isometric view with exposed interior components of a track head fluid dispenser.

FIG. 52A illustrates a view of the distal end of a particle applicator.

FIG. 52B is a side view of the delivery device of FIG. 52A applying particles to the skin.

FIG. 52C is a side view of an applicator used to disperse the droplets provided in FIG. 52B.

FIG. 53A illustrates a container of pads pre-moistened with a particle formulation.

FIG. 53B is an isometric view of an applicator used in conjunction with a pad from FIG. 53A to deliver particles to the skin.

FIG. 54 is an isometric view of an adhesive particle delivery pouch.

FIG. 55 is an isometric view of an adhesive particle delivery patch having multiple individual particle formulation capsules.

FIG. 56A is an isometric view of a set of pre-shaped adhesive particle delivery pads.

FIG. 56B illustrates the use of an applicator to deliver particles from a pad in FIG. 56A.

FIG. 57A is an isometric view of a set of pre-shaped adhesive particle delivery pad shaped for the perimeter of the face.

FIG. 57B illustrates the use of an applicator to deliver particles from a pad in FIG. 57A.

FIG. 58A is an isometric view of a set of pre-shaped adhesive particle delivery pad shaped for the face.

FIG. 58B illustrates the use of an applicator to deliver particles from a pad in FIG. 58A.

FIG. 59A illustrates a plurality of frozen particle formulations.

FIG. 59B illustrates one of the particle formulations of FIG. 59B prior to insertion into a suitable applicator used to liquefy the particle formation for penetration into skin target sites.

FIG. 60 is an isometric view of a suction based dispenser and applicator.

FIG. 61 is an isometric view of a trigger activated rotating massage applicator.

FIG. 62 is an isometric view of a palm held applicator with a fingertip activated fluid dispenser.

FIGS. 63A and 63B are isometric views of a dual sided dispenser and applicator with the fluid dispenser down (FIG. 63A) and the massager down (FIG. 63B).

FIG. 63C is an end view of FIG. 63A.

FIG. 64 is an isometric view of an alternative wand based design.

FIG. 65 is a dispenser having an air cartridge assisted delivery configuration.

FIG. 66 is an isometric view of a trigger activated hand held applicator prior to insertion of a disposable fluid cartridge.

FIG. 67 is an isometric hand held massager and fluid delivery device.

FIG. 68 is an isometric view of a hand held massager and fluid delivery device that covers less of the hand than the embodiment of FIG. 67.

FIG. 69 is an isometric view of a rolling massager and fluid dispenser.

FIG. 70 is an isometric view of a dual ended dispenser.

FIG. 71 is an isometric view of another hand held applicator with separate drive and power system as illustrated above in FIGS. 14A-15B.

FIG. 72 is a side view of a spinning dispenser pad.

FIG. 73 is a side view of the distal end of an applicator using a combination of suction and vibration for particle delivery.

FIG. 74 is a section view of the distal end of an applicator using piezoelectric vibration for particle delivery.

FIG. 75 is a section view of the distal end of an applicator using induction coils and a magnet for particle delivery.

FIG. 76 is a view of a distal end of an applicator having an alternative vibration device.

FIG. 77 is an isometric view of an applicator with two rotating heads.

FIGS. 78A and 78B are isometric and side views of an applicator with three rotating heads.

FIG. 79A illustrates a side view of an applicator having two vibrating spheres. FIG. 79B illustrates the applicator in FIG. 79A in use on a nose.

FIG. 80 illustrates an isometric and partial section view of the distal end of an applicator having a plurality of horizontal rollers.

FIG. 81 is a section view of the distal end of an applicator having a plurality of individually spring loaded fingers.

FIGS. 82A and 82B are, respectively, isometric and side views of applicator having individual pins that move via interaction with a rotating loaded ramp.

### DETAILED DESCRIPTION

When a feature or element is herein referred to as being “on” another feature or element, it can be directly on the other feature or element, or intervening features and/or elements may also be present. In contrast, when a feature or element is referred to as being “directly on” another feature or element, there are no intervening features or elements present. It will also be understood that, when a feature or element is referred to as being “connected”, “attached” or “coupled” to another feature or element, it can be directly connected, attached or coupled to the other feature or element, or intervening features or elements may be present. In contrast, when a feature or element is referred to as being “directly connected”, “directly attached” or “directly coupled” to another feature or element, there are no intervening features or elements present. Although described or shown with respect to one embodiment, the features and elements so described or shown can apply to other embodiments. It will also be appreciated by those of skill in the art that references to a structure or feature that is disposed “adjacent” another feature may have portions that overlap or underlie the adjacent feature.

Terminology used herein is for the purpose of describing particular embodiments only and is not intended to be limiting of the invention. For example, as used herein, the singular forms “a”, “an” and “the” are intended to include the plural forms as well, unless the context clearly indicates otherwise. It will be further understood that the terms “comprises” and/or “comprising.” when used in this specification, specify the presence of stated features, steps, operations, elements, and/or components, but do not preclude the presence or addition of one or more other features, steps, operations, elements, components, and/or groups thereof. As used herein, the term “and/or” includes any and all combinations of one or more of the associated listed items and may be abbreviated as “/”.

Spatially relative terms, such as “under”, “below”, “lower”, “over”, “upper” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if a device in the figures is inverted, elements described as “under” or “beneath” other elements or features would then be oriented “over” the other elements or features. Thus, the exemplary term “under” can encompass both an orientation of over and under. The device may be otherwise oriented (rotated 90 degrees or at other orientations) and the spatially relative descriptors used herein interpreted accordingly. Similarly, the terms “upwardly”, “downwardly”, “vertical”, “horizontal” and the like are used herein for the purpose of explanation only unless specifically indicated otherwise.

Although the terms “first” and “second” may be used herein to describe various features/elements, these features/elements should not be limited by these terms, unless the context indicates otherwise. These terms may be used to distinguish one feature/element from another feature/element. Thus, a first feature/element discussed below could be termed a second feature/element, and similarly, a second feature/element discussed below could be termed a first feature/element without departing from the teachings of the present invention.

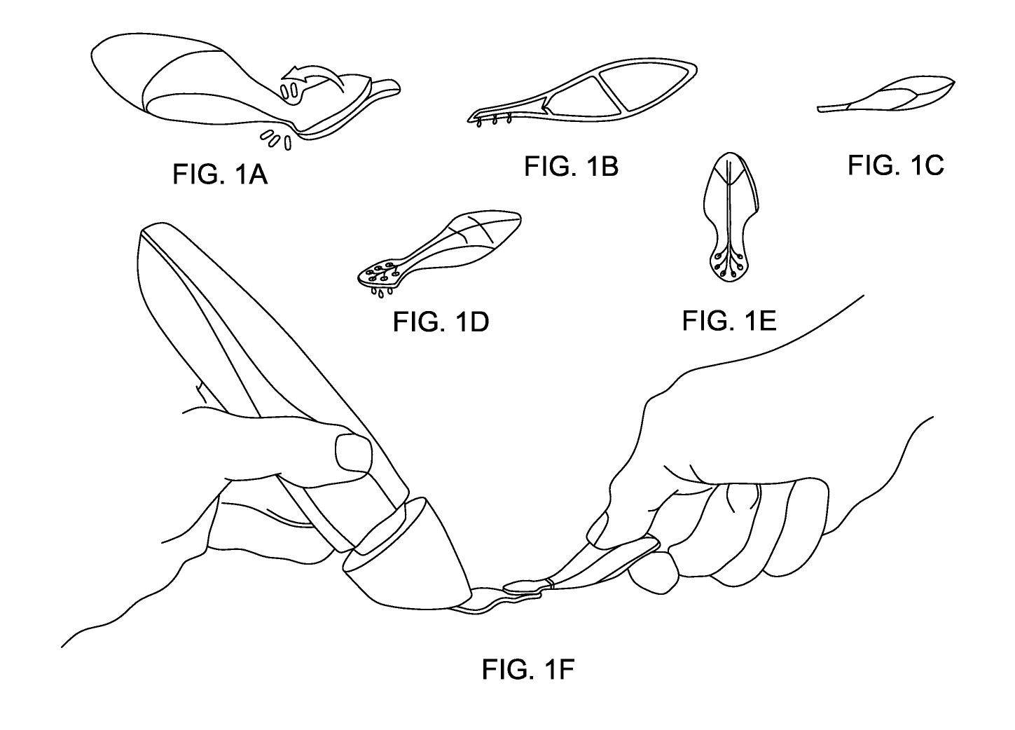

FIGS. 1A-1F illustrate various views of a separate particle fluid dispenser pack and use with an exemplary applicator (FIG. 1F).

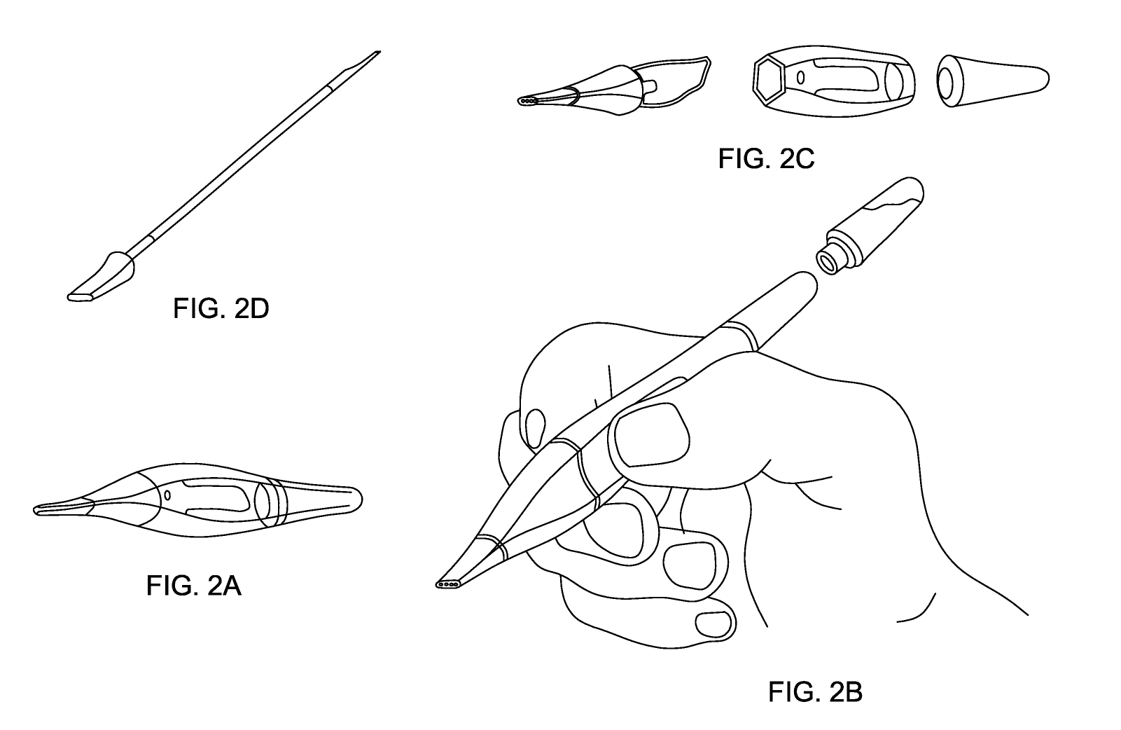

FIGS. 2A and 2B illustrate isometric and in use views, respectively, of an applicator pen. FIG. 2C is an exploded view and FIG. 2D is an alternative configuration.

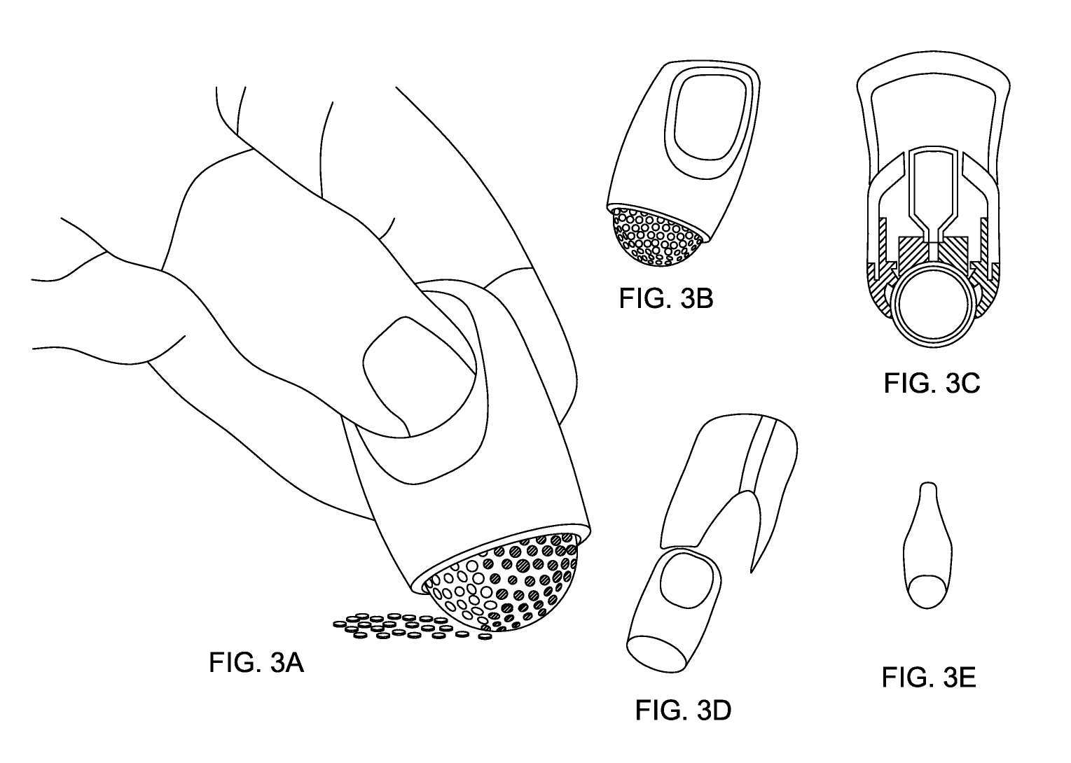

FIGS. 3A, 3B and 3C illustrate in use, side and cross section views, respectively, of a roller ball applicator. FIGS. 3D and 3E provide alternative views.

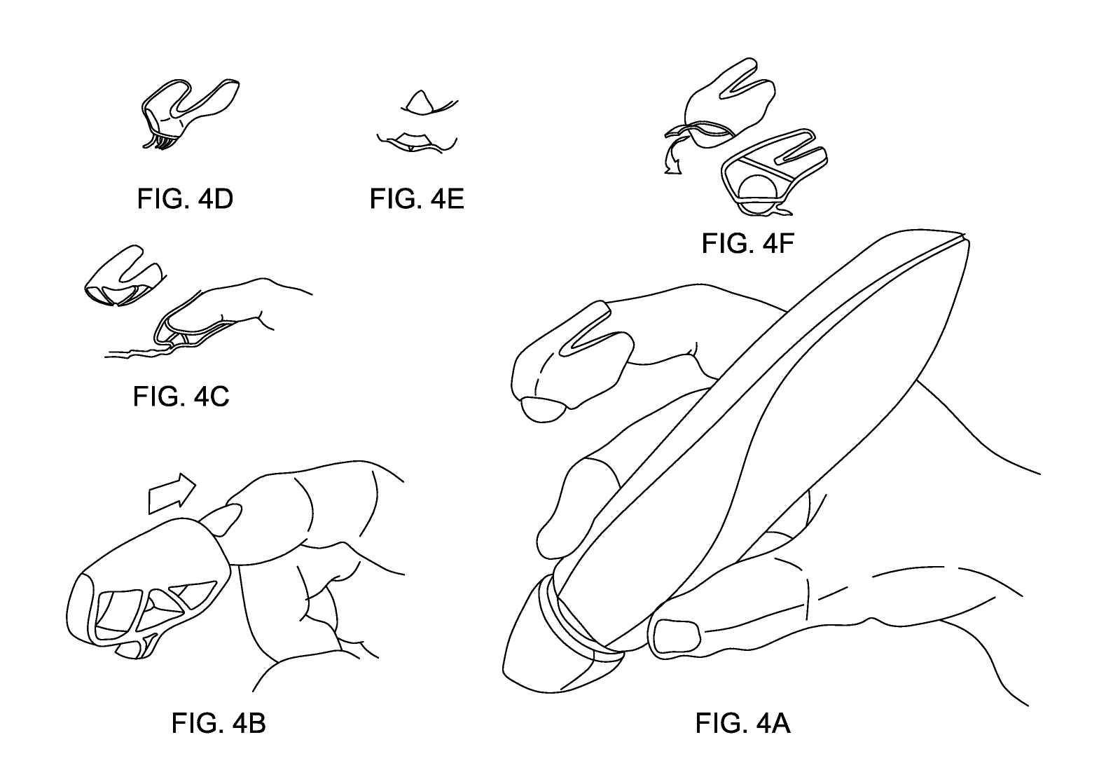

FIG. 4A illustrates an in use, isometric view of a fingertip dispenser and hand held applicator. FIG. 4B is a cross section view of an alternative fingertip dispenser embodiment. FIGS. 4C-4F are additional views of alternative fingertip dispenser embodiments.

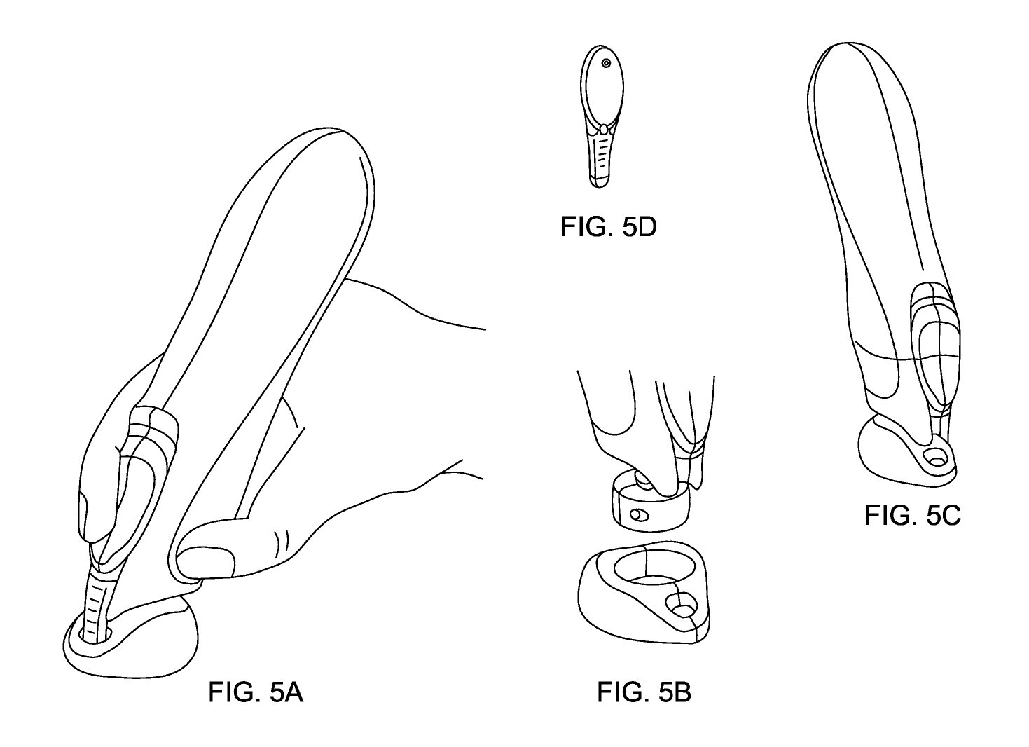

FIG. 5A illustrates an in use, isometric view of a disposable delivery container integrated into a hand held applicator. FIG. 5B is an isometric view of the treatment head before attachment to the applicator and attached in FIG. 5D. FIG. 5C is the particle container before attachment to the applicator as shown in FIG. 5A.

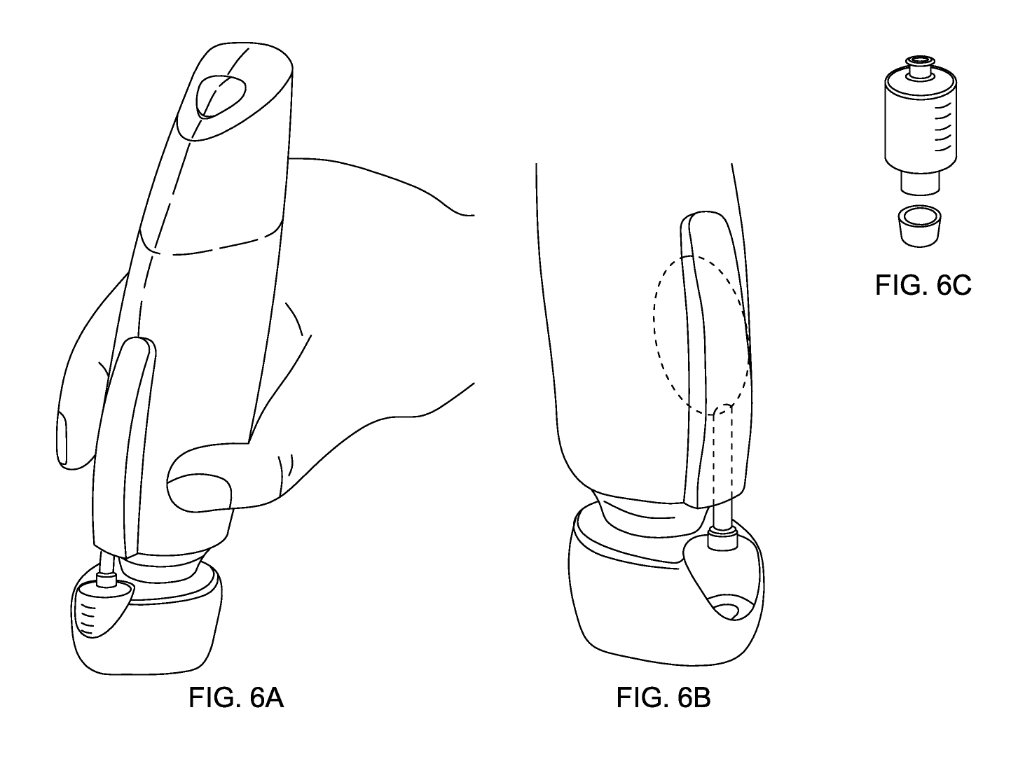

FIG. 6A illustrates an in use, isometric view of a disposable delivery container integrated into a hand held applicator. FIG. 6B is an isometric view of the applicator before insertion of the particle container. FIG. 6C is an isometric view of the particle container shown n FIG. 6A.

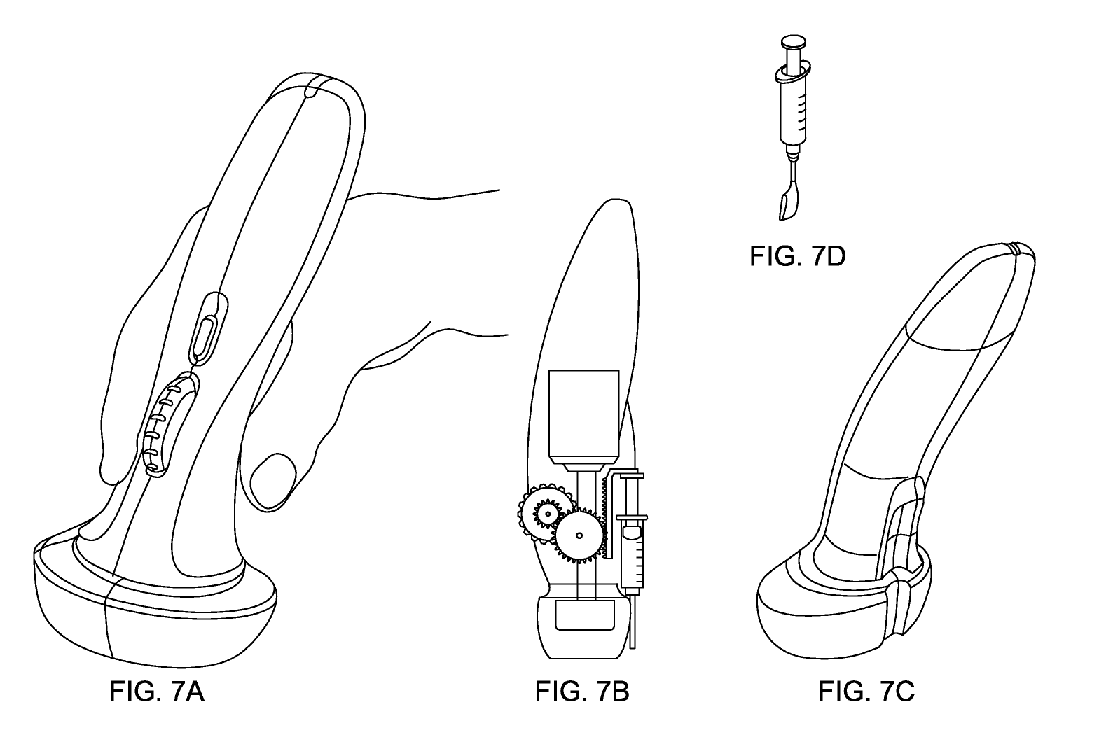

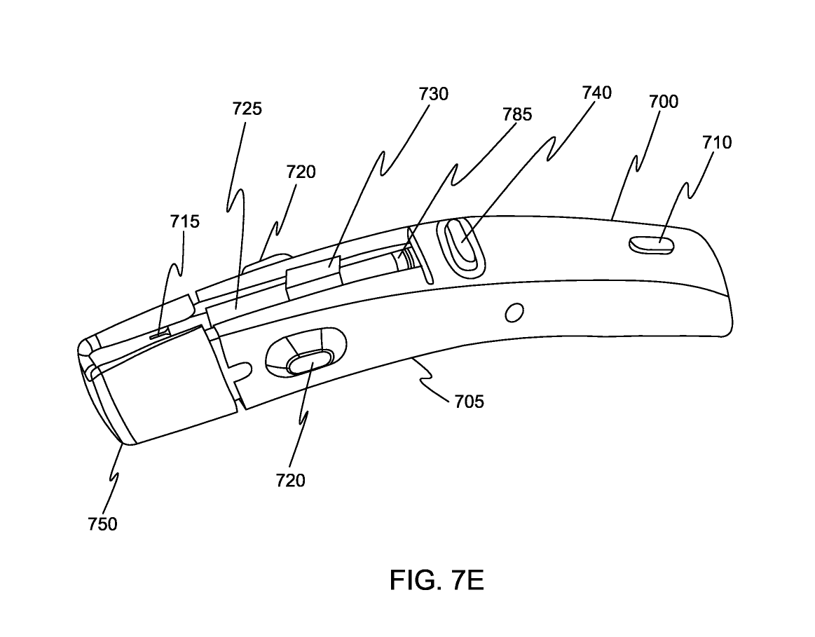

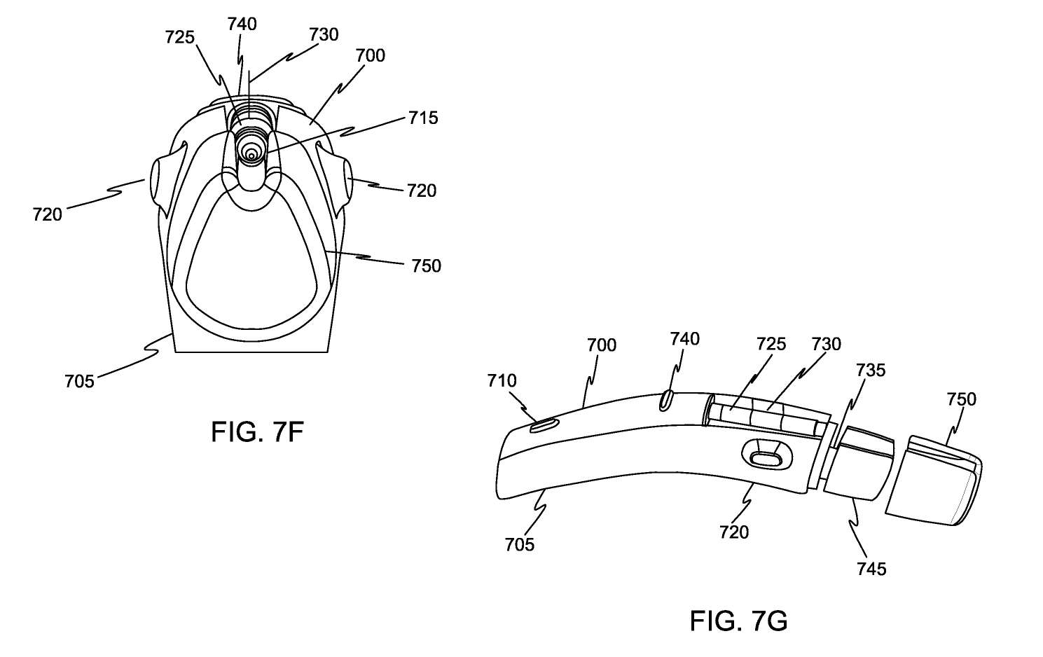

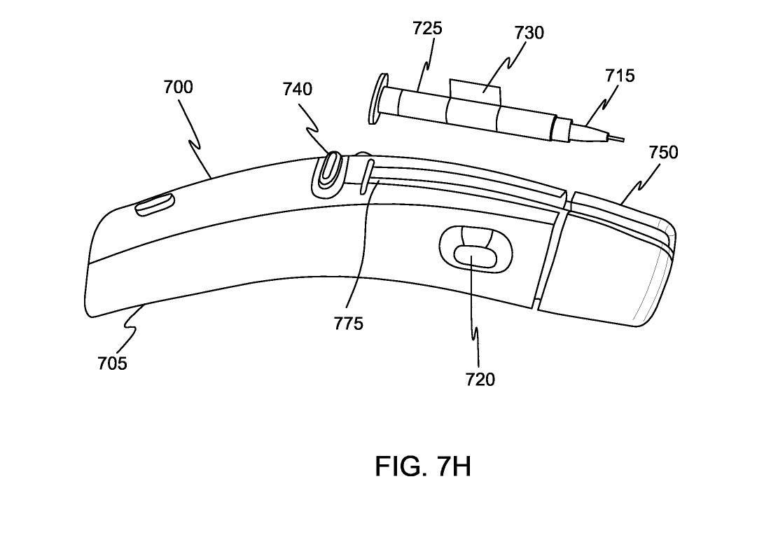

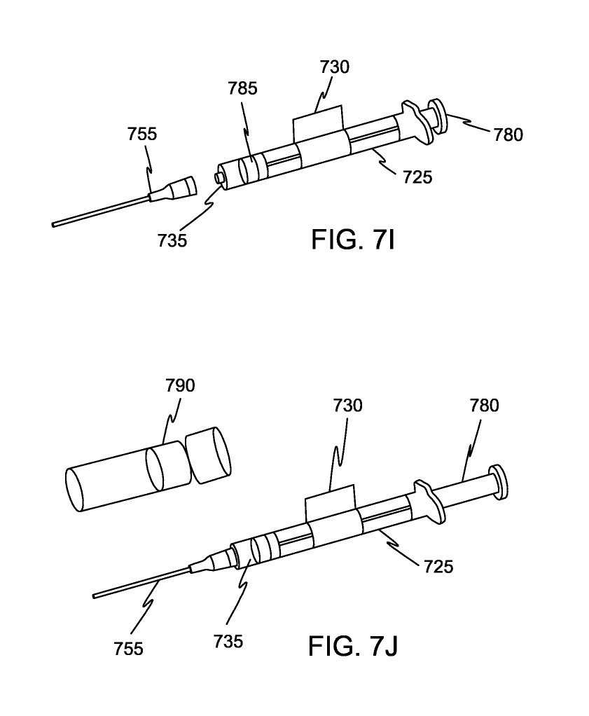

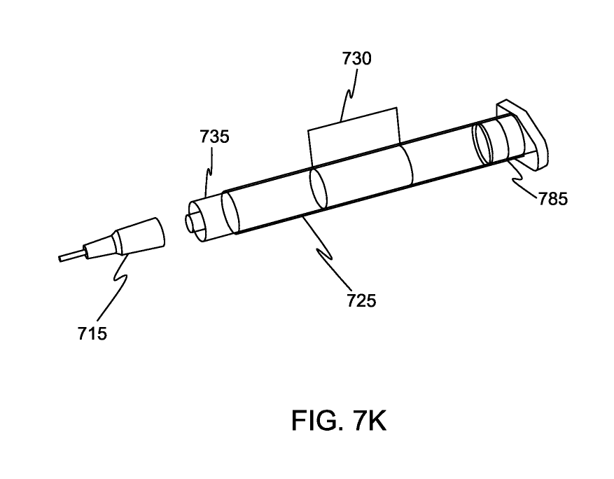

FIG. 7A illustrates an in use, isometric view of a disposable delivery container integrated into a hand held applicator. FIG. 7B is a section view of the application in FIG. 7A illustrating the rack and pinion drive mechanism with a disposable delivery container integrated into a hand held applicator. FIG. 7C illustrates a rear view of the applicator of FIG. 7A. FIG. 7D is an isometric view of the particle container shown in FIG. 7B.

Tuning to embodiment 700 of a device of the present invention illustrated in FIGS. 7 E through K, the device has a body 705. Within body 705, but not illustrated, is a conventional battery operated motor and battery. Also within body 705, and also not seen, is a conventional mechanism, coupled to said motor, that moves the head of device 700, covered by head cover 750, in a manner that massages a surface that is in contact with head cover 750.

FIG. 7 E also shows power button 710, disposable delivery container 725, pull tab 730, release button 740, delivery tip 715, stopper 785 and actuator buttons 720.

FIG. 7 F shows a front view of embodiment 700 of a device of the present invention illustrated in FIGS. 7 E through K. From this perspective, head cover 750 is nearest the viewer. Within a space in head cover 750 is delivery tip 715 through which the composition containing particles for delivery to a subject's skin (for example, a composition containing plasmonic nanoparticles) are delivered. On each side of body 705 are actuator buttons 720. Projecting up from disposable delivery container 725 is pull tab 730, and behind disposable delivery container 725 is release button 740.

FIG. 7 G provides another side view of embodiment 700 of a device of the present invention illustrated in FIGS. 7 E through K. Again, the device has a body 705. FIG. 7 F also shows power button 710, disposable delivery container 725, pull tab 730, release button 740, actuator buttons 720, and head cover 750. However, in this view, head cover 750 is not covering device head 745.

Also shown in FIG. 7 G is connector 735 which transmits the massaging action from a motor within body 705 to device head 745.

FIG. 7 H provides a still further side view of embodiment 700 of a device of the present invention illustrated in FIGS. 7 E through K. In this view, disposable delivery container 725 with pull tab 730 and delivery tip 715 are positioned above, and not within, embodiment 700. Within body 705 below disposable delivery container 725 is a receptacle 775 for receiving disposable delivery container 725.

FIG. 7 H also shows release button 740, actuator buttons 720, and head cover 750.

FIG. 7 I shows an embodiment of a delivery container 725 for use in an embodiment 700 of a device of the present invention illustrated in FIGS. 7 E through K. In this view, delivery container 725 has a removable plunger 780 attached to stopper 785, and both are advanced to the delivery end of the delivery container and next to luer connector 735. Pull tab 730 projects from delivery container 725 and filling tip 755 is shown disengaged in front of luer connector 735.

FIG. 7 J shows an embodiment of a delivery container 725 for use in an embodiment 700 of a device of the present invention illustrated in FIGS. 7 E through K. In this view, delivery container 725, removable plunger 780 is shown partially withdrawn (and partially hidden by pull tab 730) and filling tip 755 is shown connected to delivery container 725 via luer connector 735. FIG. 7 J also shows filling vial 790.

FIG. 7 K shows an embodiment of a delivery container 725 for use in an embodiment 700 of a device of the present invention illustrated in FIGS. 7 D through K. In this view, delivery container 725, the removable plunger (not shown) has been detached from stopper 785, which is in a fully retracted position. Additionally, the filling tip has been removed (not shown), and delivery tip 715 is positioned next to the luer connector 735 for attachment to the delivery container 725. Pull tab 730 again projects from delivery container 725.

The device of the present invention illustrated in FIGS. 7 E through K can be used by first filling the disposable delivery container 725. For instance, a fluid to be dispensed can be prepared in filling vial 790. Removable plunger 780 is secured to stopper 785, typically by screwing a distal portion of removable plunger 780 into the stopper. Additionally, filling tip 755 is secured to luer connector 735. Stopper 785 is advanced to the delivery end of delivery container 725 by pushing on removable plunger 780. Filling tip 755 is positioned in filling vial 790, and removable plunger 780 is withdrawn and the fluid to be dispensed is drawn into the delivery container 725. When an appropriate volume of the fluid is within delivery container 725 (in a preferred embodiment delivery container 725 is marked with graduations indicating the approximate volume of fluid within delivery container 725), filling tip 755 is removed from filling vial 790 and detached from luer connector 735. At about the same time, removable plunger 780 is detached from stopper 785, typically by unscrewing.

Once delivery container 725 has been filed, delivery tip 715 is positioned next to the luer connector 735 and attached to delivery container 725.

Filled delivery container 725 without removable plunger 780, but with delivery tip 715, generally by being held by pull tab 730, is positioned over, and inserted into, receptacle 775. Once filled delivery container 725 is within receptacle 775, actuator buttons 720 are squeezed toward the center of device body 705. Each time actuator buttons 720 are squeezed, a plunger (not shown) within device body 705 is advanced toward, and into delivery container 725. Once the plunger within device body 705 contacts stopper 785, each time actuator buttons 720 are squeezed, stopper 785 is advanced so as to dispense approximately 0.1 ml of fluid per increment from delivery container 725.

Once the plunger within device body 705 is positioned to advance stopper 785 to dispense a reproducible small volume, generally between about 0.1 and 0.3 ml of fluid, from delivery container 725 with each squeeze of actuator buttons 720, the device is ready for use.

Releasing, after previously depressing, power button 710 activates the internal battery powered motor which moves the head of device 700 in a manner that imparts a massaging action to skin in which the head cover 750 is in contact. Thus, the user of device 700, holding the device in contact with the portion of a subject's skin to be treated with the fluid being dispensed, is massaging the skin. At intervals determined by the user, actuator buttons 720 are squeezed, the fluid is dispensed, and the subject's skin to which the fluid was applied is massaged. This massage helps facilitate the delivery of material in the fluid into structures such as follicles in the skin.

After the desired volume of the composition containing particles for delivery to a subject's skin has been delivered, delivery container 725 is removed from device body 705. Typically, the removal process begins with depressing release button 740, which activates a mechanism that withdraws the plunger within device body 705. At that point, delivery container 725 is removed from receptacle 775 by pulling pull tab 730 away from body 705.

It is further envisioned that head cover 750 will be replaced with a new, single use, head cover 750 before the device is used to deliver a composition to another subject.

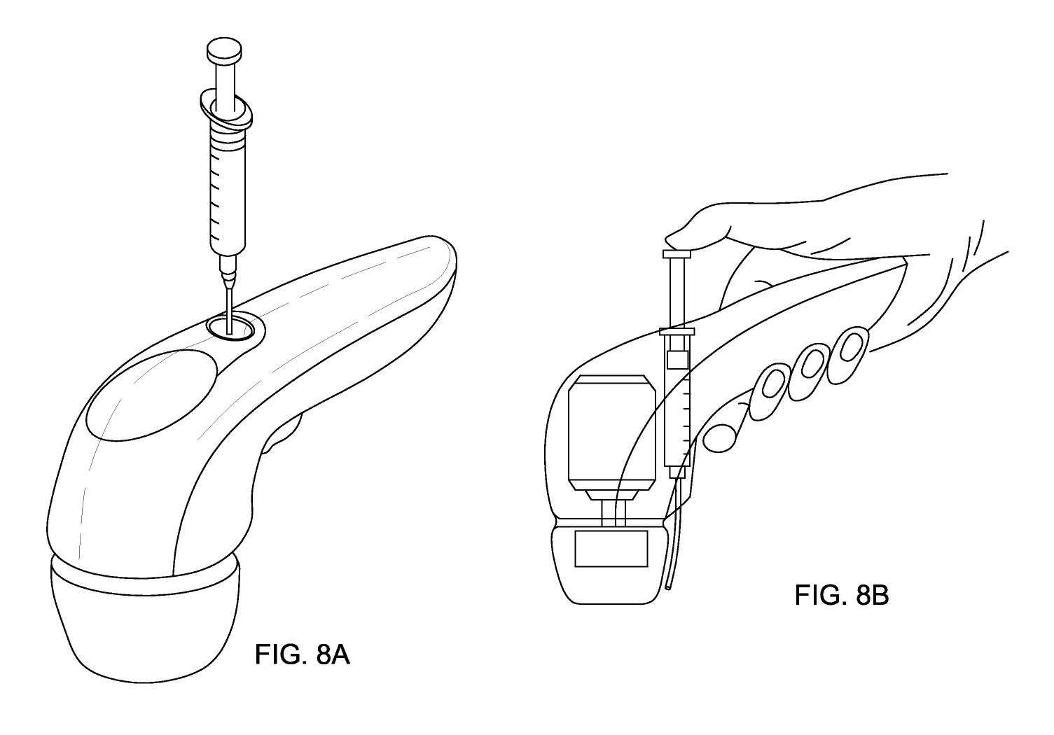

FIG. 8A illustrates an isometric view of a disposable delivery container prior to insertion into a hand held applicator. FIG. 8B is a section view of the applicator and inserted container of FIG. 8B.

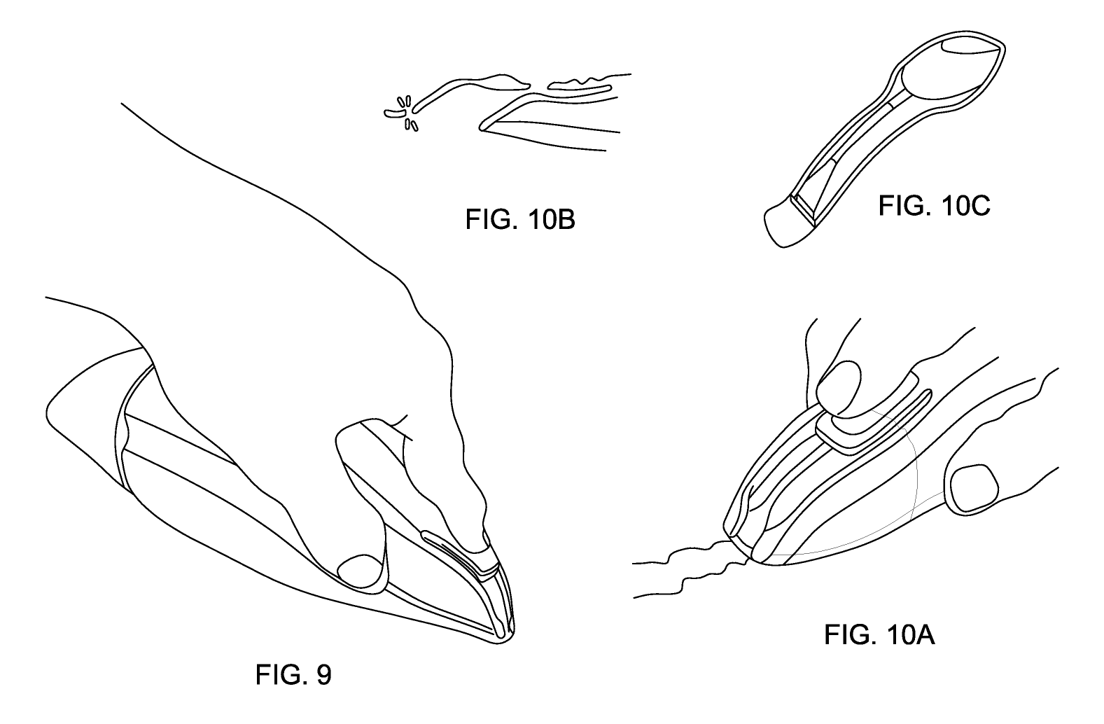

FIG. 9 illustrates an isometric view of a dual ended hand held applicator.

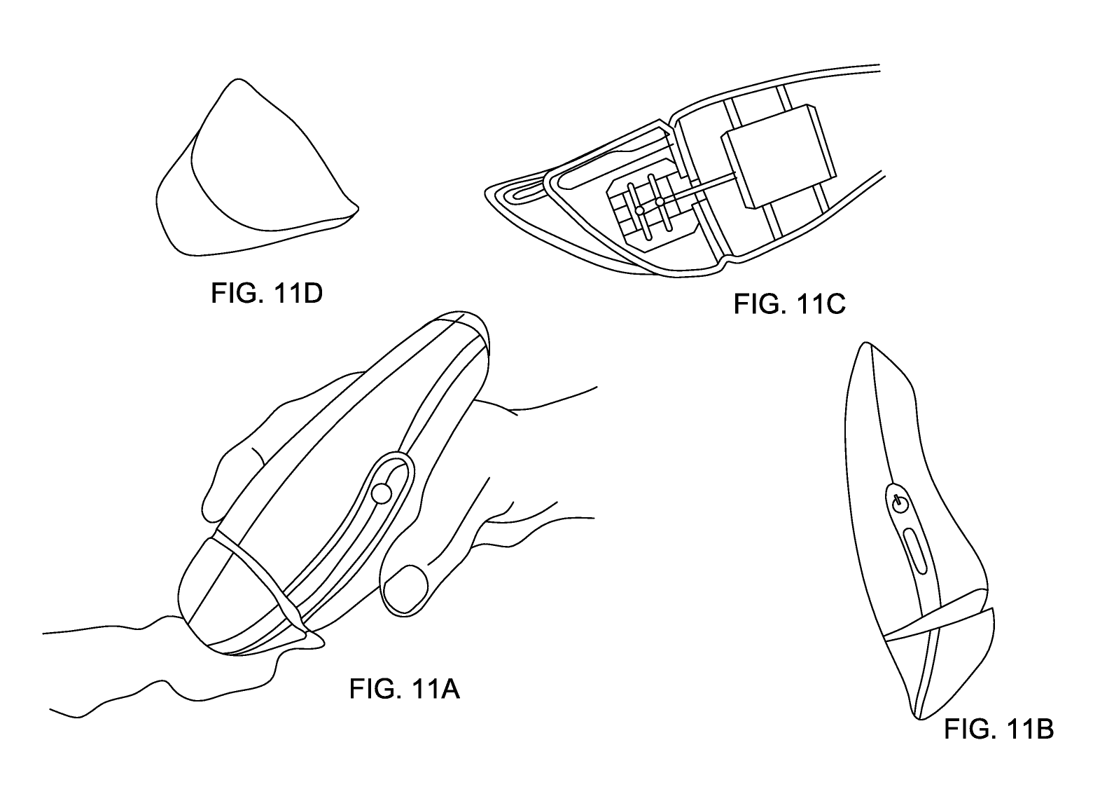

FIG. 10A illustrates an enlarged view of the dispensing end of a dual ended applicator with a particle container in place. FIG. 10B illustrates the insertion of a particle container into the dispensing end of a dual ended applicator. FIG. 10C is an isometric view of the particle container illustrated in FIGS. 10A and 10B.

FIGS. 11A-11D illustrate various views of an adjustable massager head.

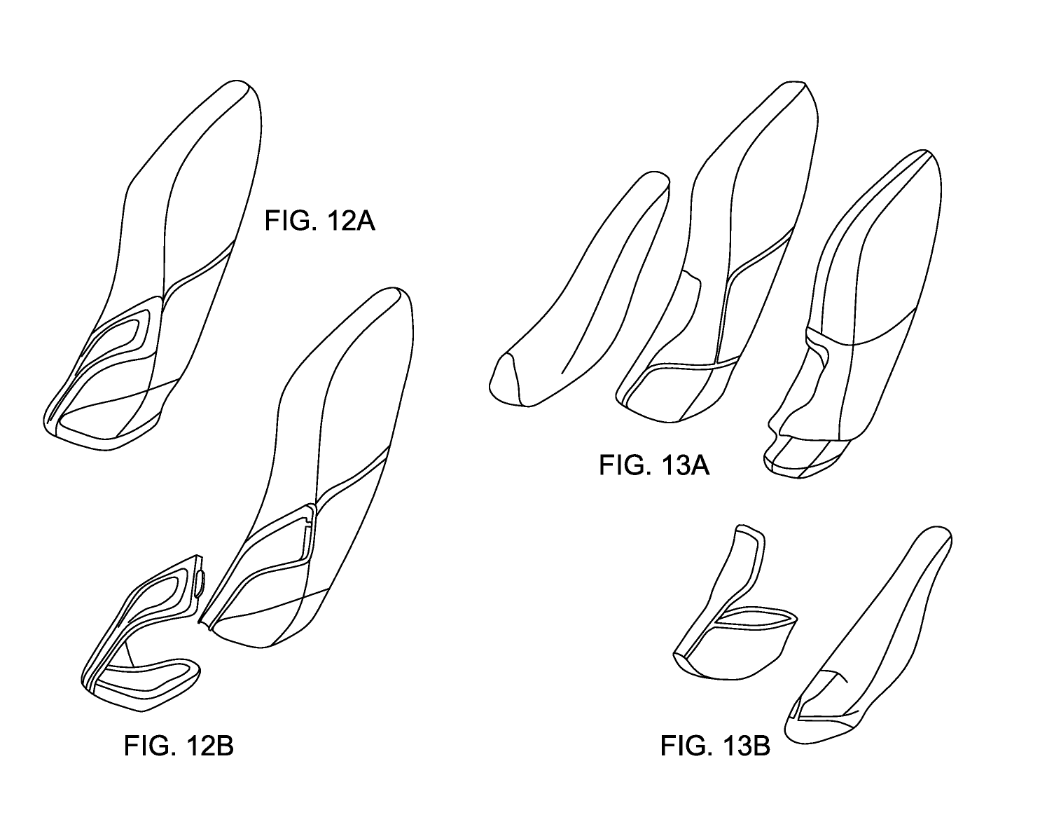

FIG. 12A illustrates an isometric view of a hand held applicator. FIG. 12B is a view of the applicator of FIG. 12A with the head removed.

FIG. 13A illustrates an isometric view of a hand held applicator. FIG. 13B is a view of the applicator of FIG. 13A with the head removed.

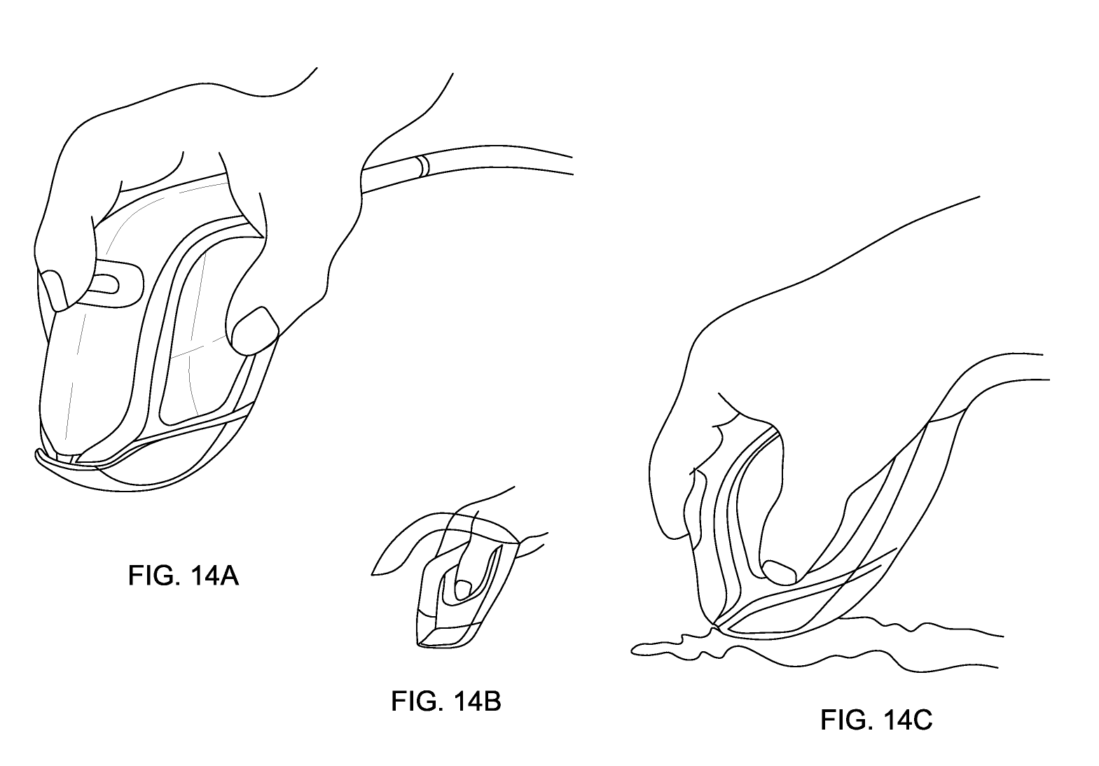

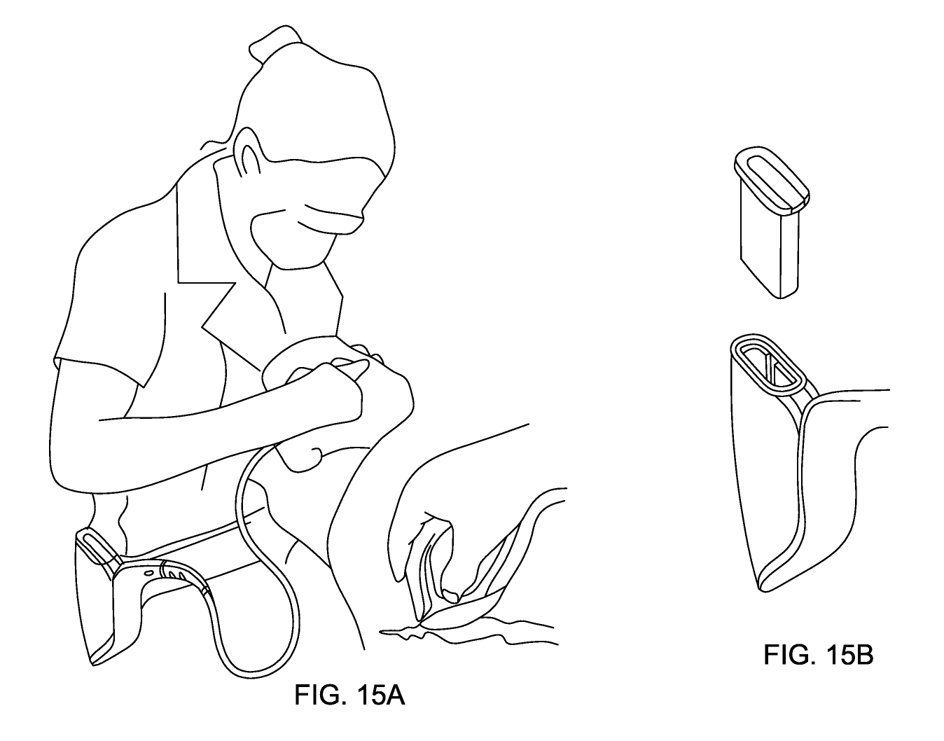

FIGS. 14A-14C illustrate various views of an palm grip dispenser.

FIG. 15A illustrates a palm dispenser of FIG. 14A-14C in use with an external battery and motor pack. FIG. 15B illustrates the removal of the battery back.

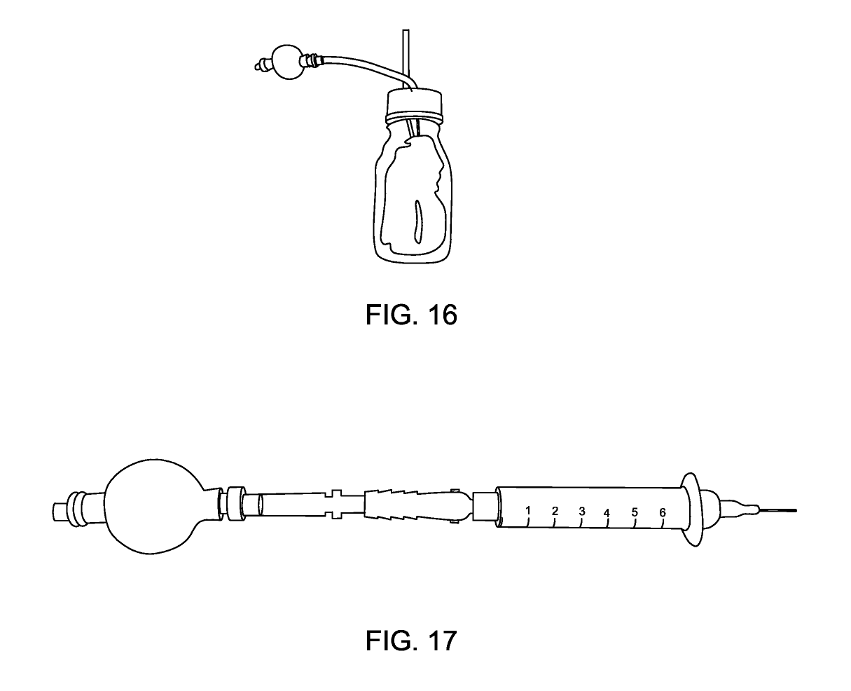

FIG. 16 is an isometric view of a delivery container having a collapsible bag within a rigid casing.

FIG. 17 is an isometric view of a bulb pump and syringe delivery container having a collapsible bag within a syringe body.

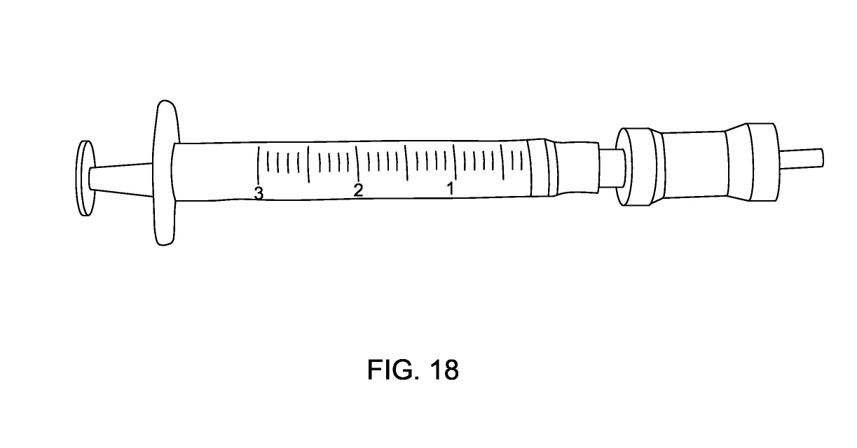

FIG. 18 is an isometric view of a syringe attached to a delivery container having a collapsible squeeze reservoir and a pair of check valves.

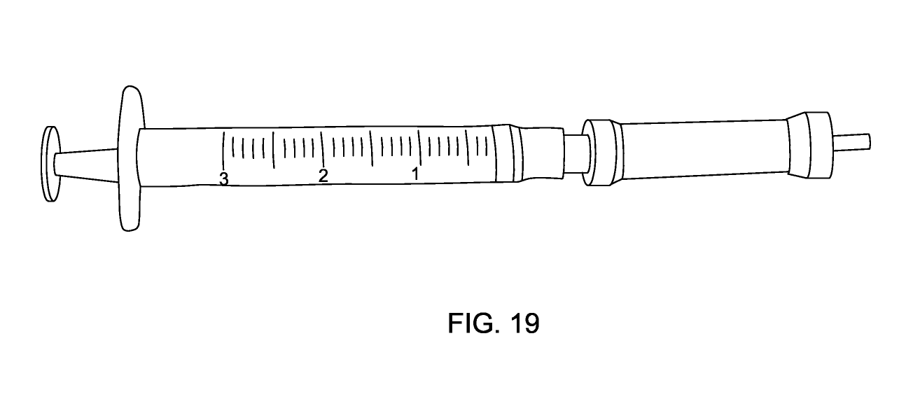

FIG. 19 is an isometric view of a syringe attached to a delivery container having a collapsible squeeze reservoir and a pair of check valves.

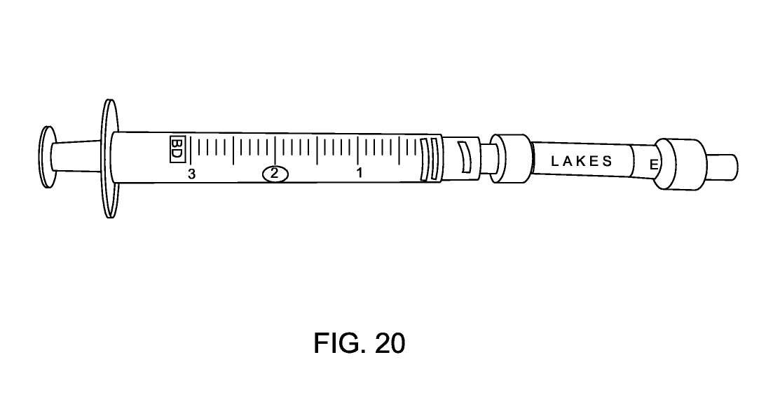

FIG. 20 is an isometric view of a syringe attached to a delivery container having a collapsible squeeze reservoir and a pair of check valves.

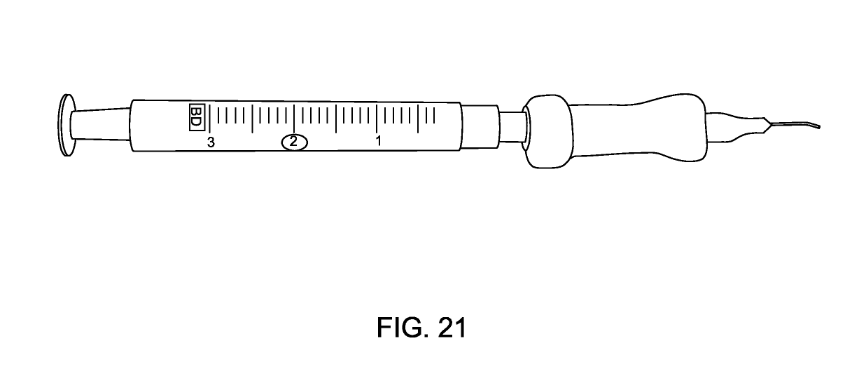

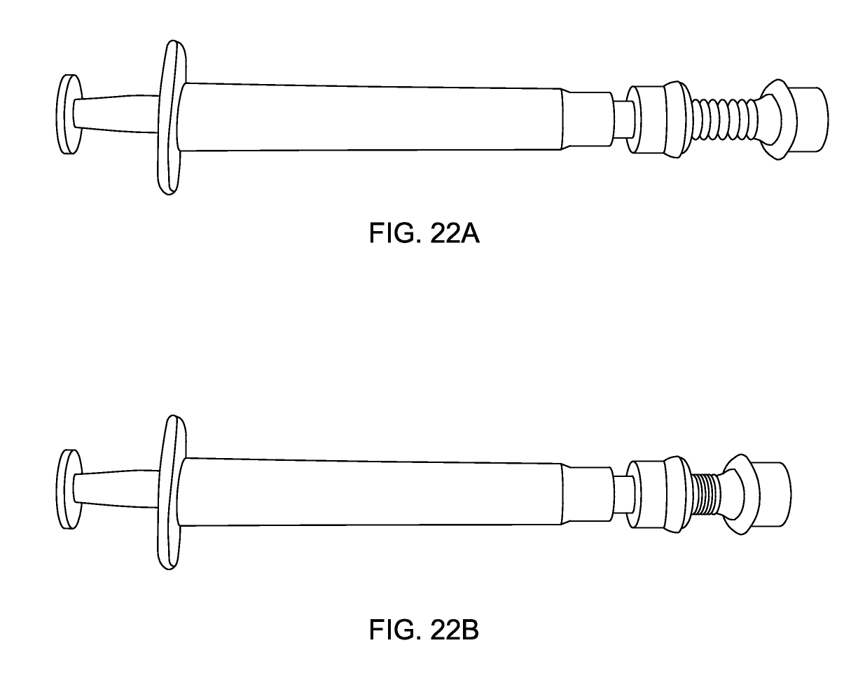

FIG. 21 is an isometric view of a syringe attached to a delivery container having a collapsible squeeze reservoir and a single check valve.

FIGS. 22A and 22B illustrate a syringe attached to a compressible bellows delivery reservoir and a pair of check valves. The bellows are shown in an expanded configuration (FIG. 22A) and compressed configuration (FIG. 22B).

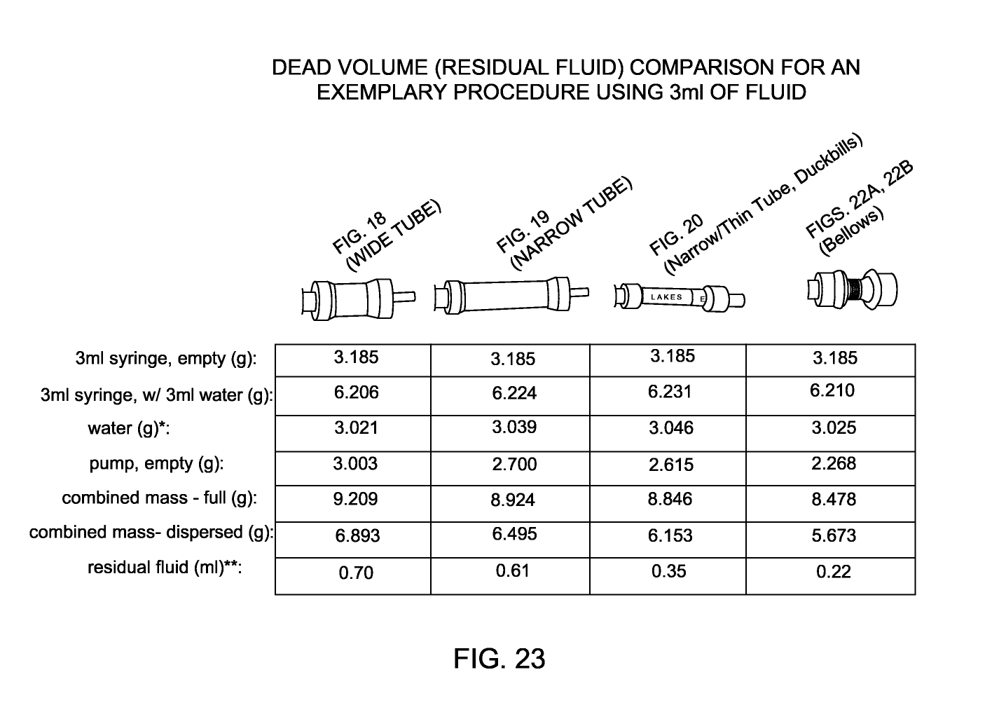

FIG. 23 is a table comparing the embodiments of FIGS. 18, 19, 20, 22A and 22B.

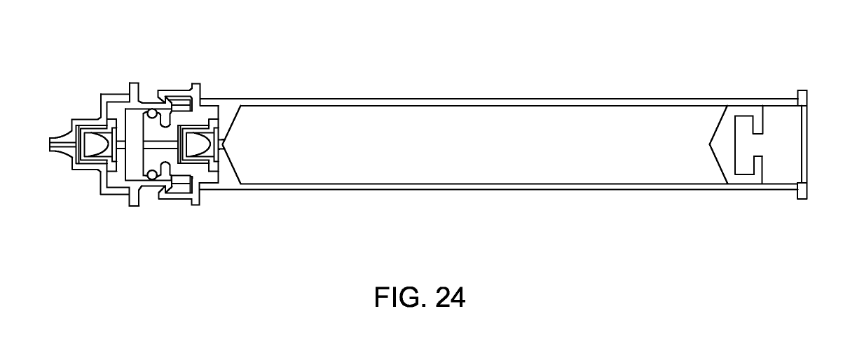

FIG. 24 is a cross section view of a piston pump fluid delivery device.

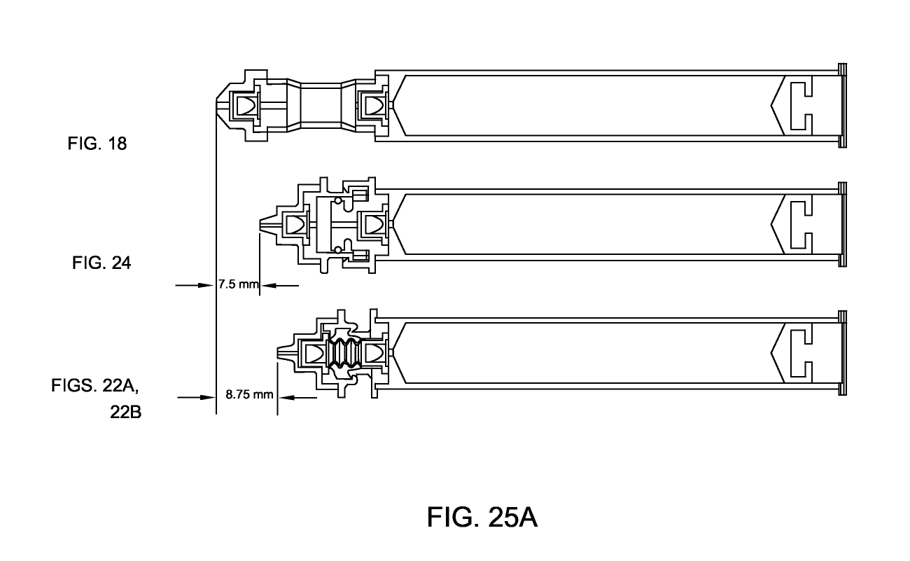

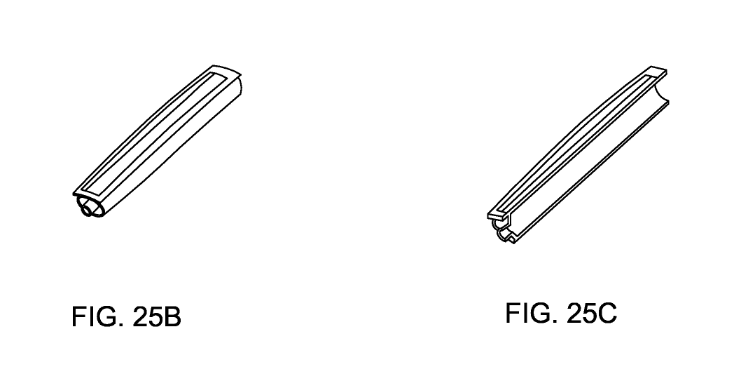

FIG. 25A is a comparison of the overall length of the various delivery devices of FIGS. 18, 24, 22A and 22B.

FIGS. 25B and 25C illustrate isometric and cross section views of an embodiment of a particle delivery container housing. FIG. 25B illustrates the exterior surface, size and shape of an exemplary particle container housing adapted and configured for use with an applicator as described herein. It is to be appreciated that the exterior features, size, shape and one or more mating features may be added depending upon the final configuration of the particle container and/or applicator. Similarly, FIG. 25C is a section view of the container in FIG. 25B. The interior detail and a suitable plunger are omitted from this view. However, it is to be appreciated that any of the various particle delivery modes described herein may be adapted and configured for use within a specifically designed particle chamber as exemplified by FIGS. 25B and 25C. In still further aspects, the particle chamber or cartridge may be adapted and configured to accommodate any of the delivery devices or modes illustrated and described in FIGS. 16-25A.

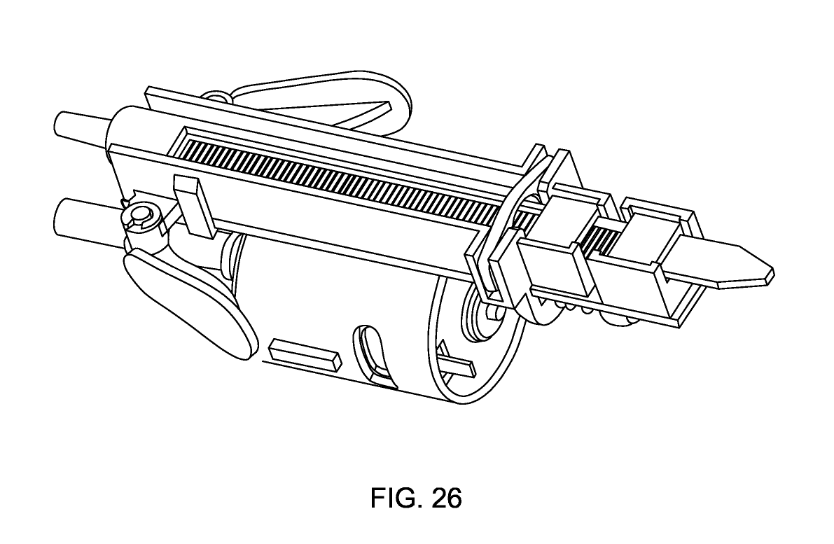

FIG. 26 is an isometric view of a syringe body with a zip tie drive system.

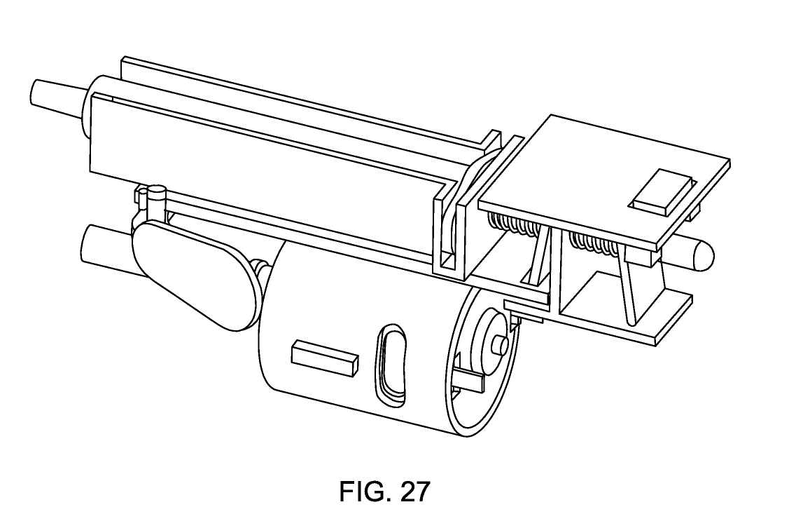

FIG. 27 is an isometric view of a syringe body with a plunger drive device.

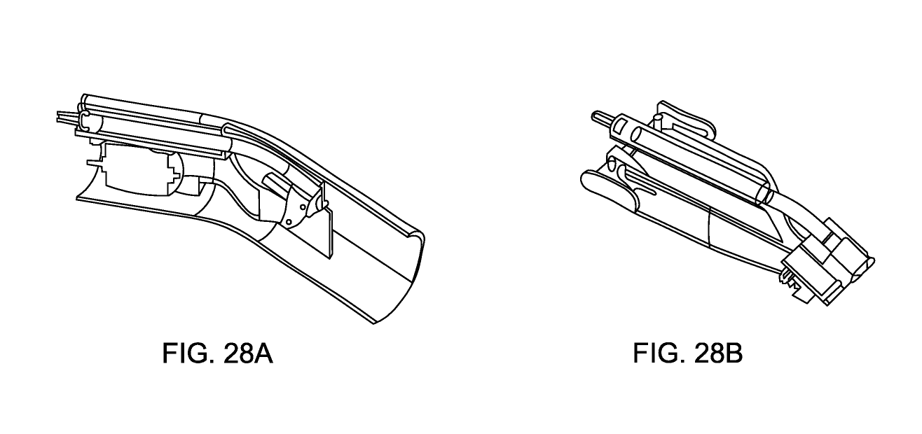

FIGS. 28A and 28B are isometric and section views of push rod delivery devices.

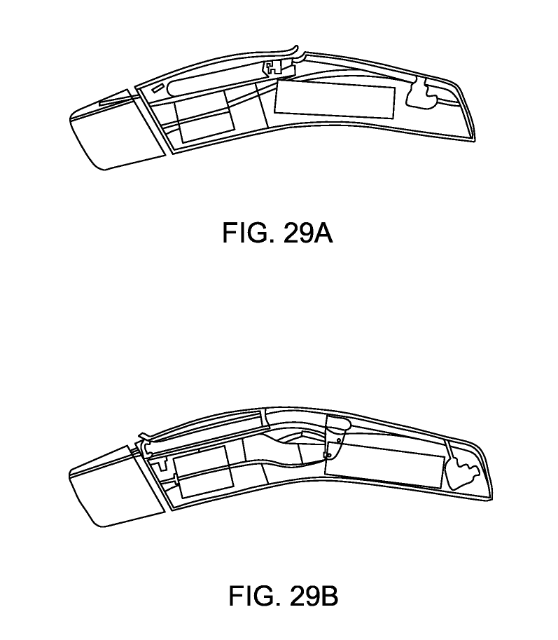

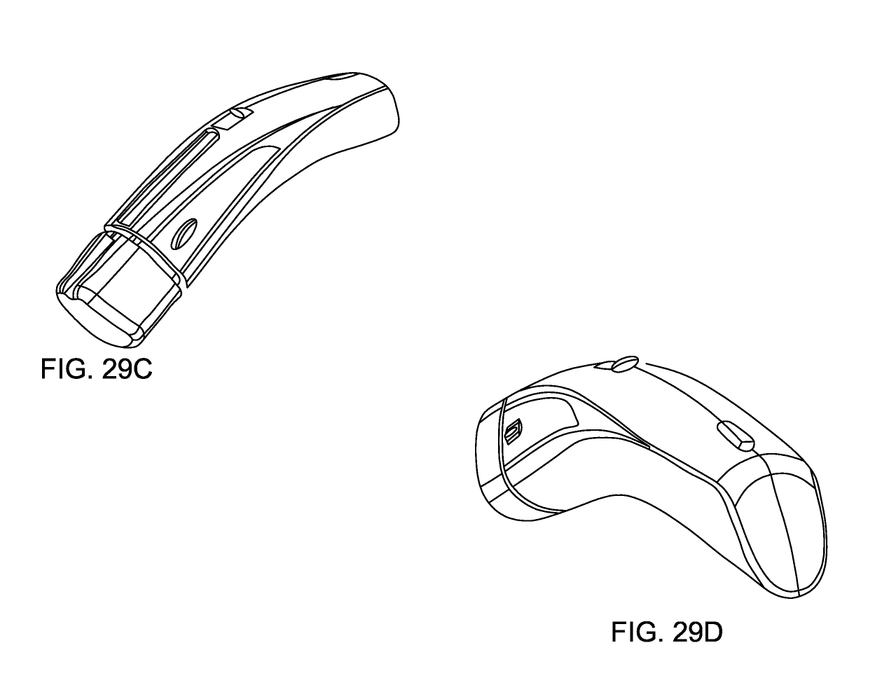

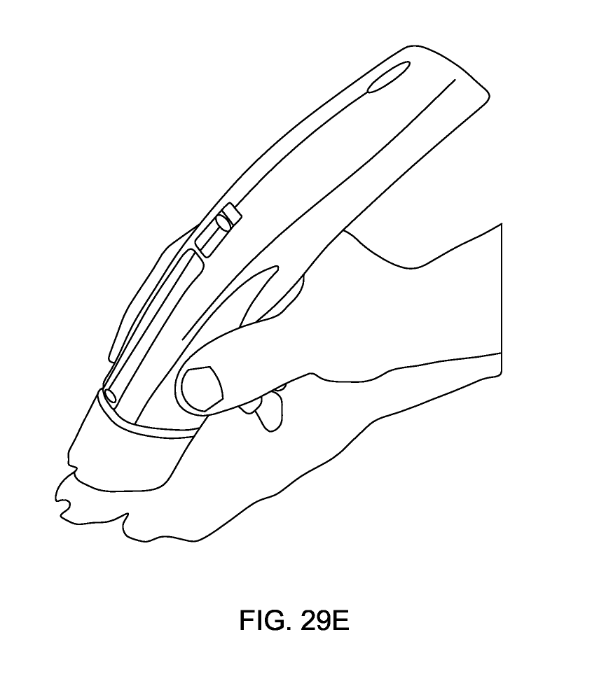

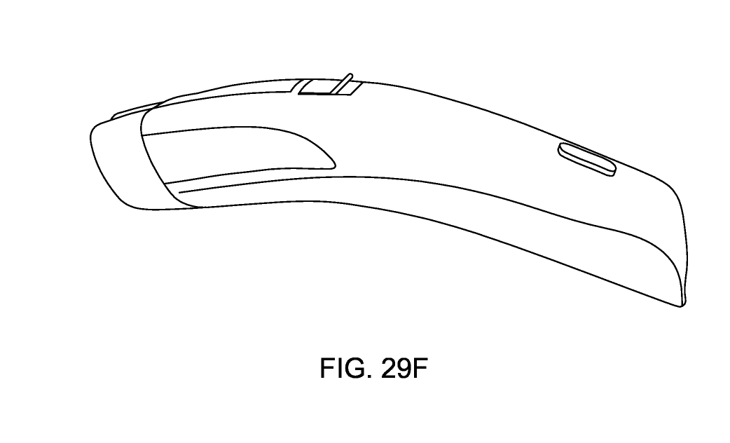

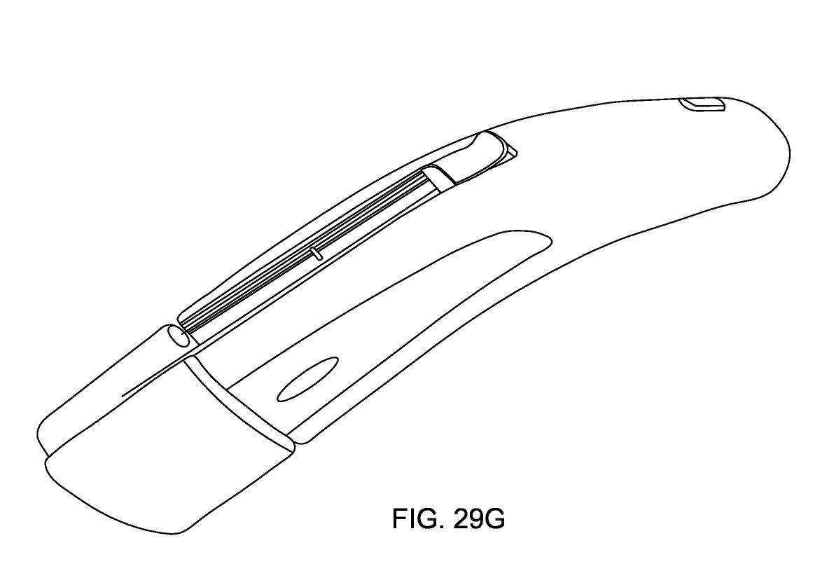

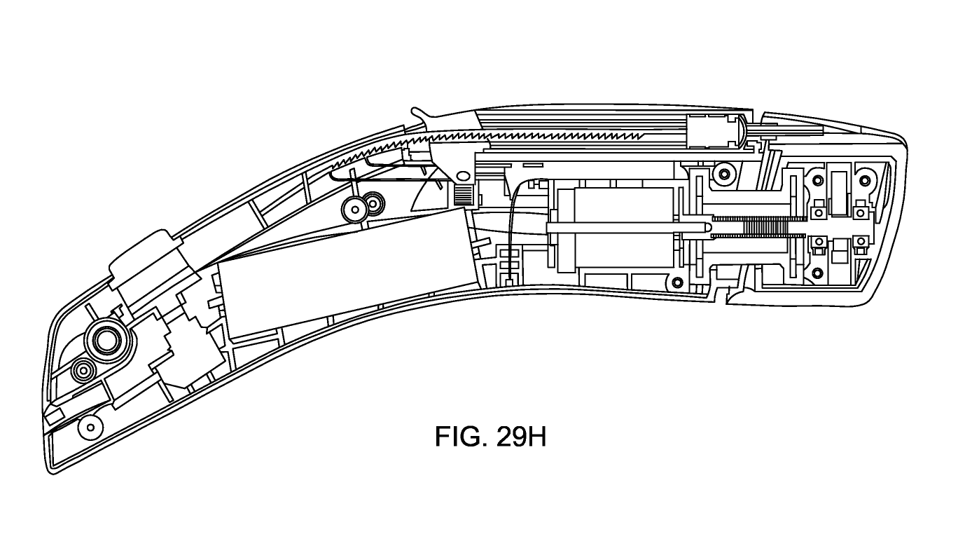

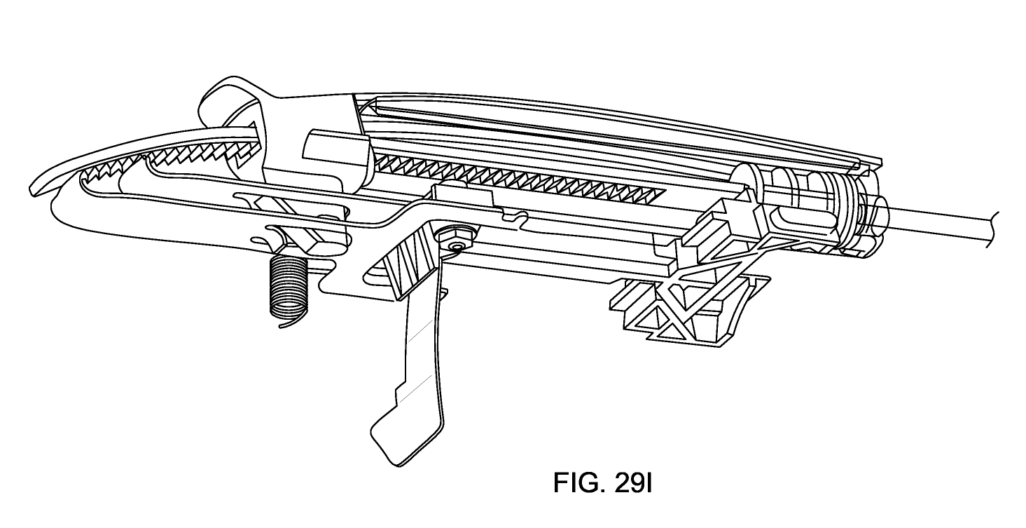

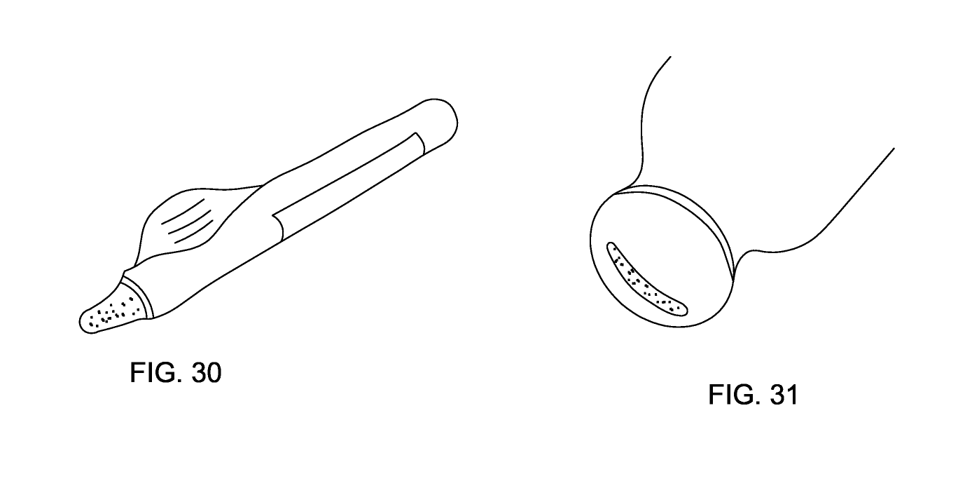

FIGS. 29A and 29B illustrate, respectively, the delivery devices of FIGS. 24 and 28A within an exemplary hand held applicator.

FIGS. 29C and 29D are side and rear isometric views, respectively of a hand held particle applicator. FIG. 29E illustrates a prototype of the applicator in FIGS. 29C and 29D showing hand hold position and orientation to treatment surface in use.

FIGS. 29F and 29G are side and isometric views, respectively of a hand held particle applicator. FIG. 29H illustrates a section view of the applicator of FIGS. 29F, 29G. FIG. 29I is an enlarged view of the plunger actuator illustrated in the section view of FIG. 29H. All or a portion of the mechanism illustrated in FIG. 29I may be modified or adapted for use with other applicators described herein, for example, in FIGS. 26-29E.

FIG. 30 is an isometric view of a fluid dispenser with a squeeze pad.

FIG. 31 is an enlarged view of the distal portion of a fluid dispenser having a material dispenser tip.

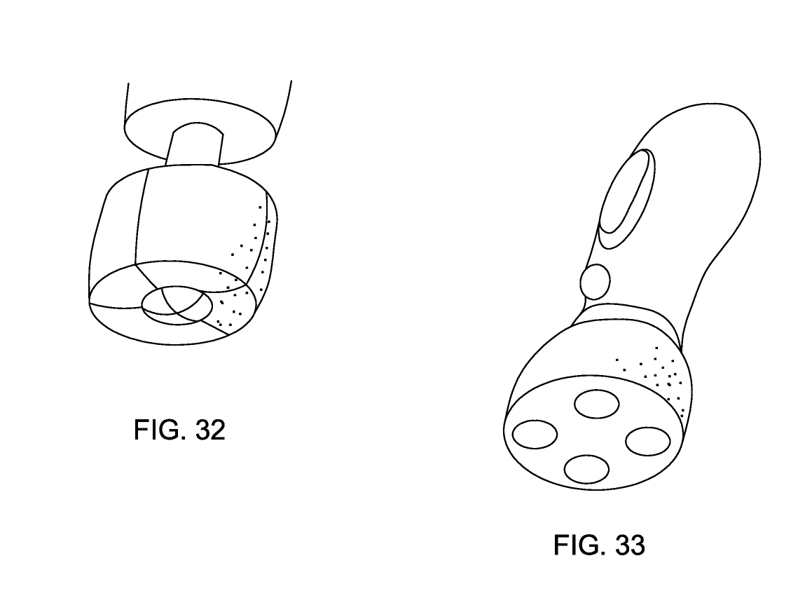

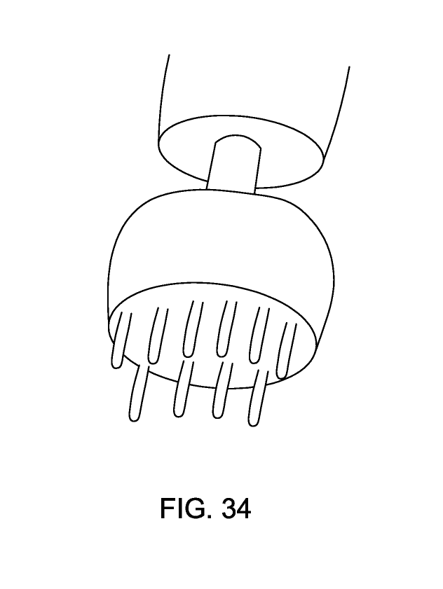

FIG. 32 is a bottom up isometric view of a single spring loaded roller ball fluid dispenser.

FIG. 33 is a bottom up isometric view of a multiple spring loaded roller ball fluid dispenser.

FIG. 34 is a bottom up isometric view of a multiple finger fluid dispenser.

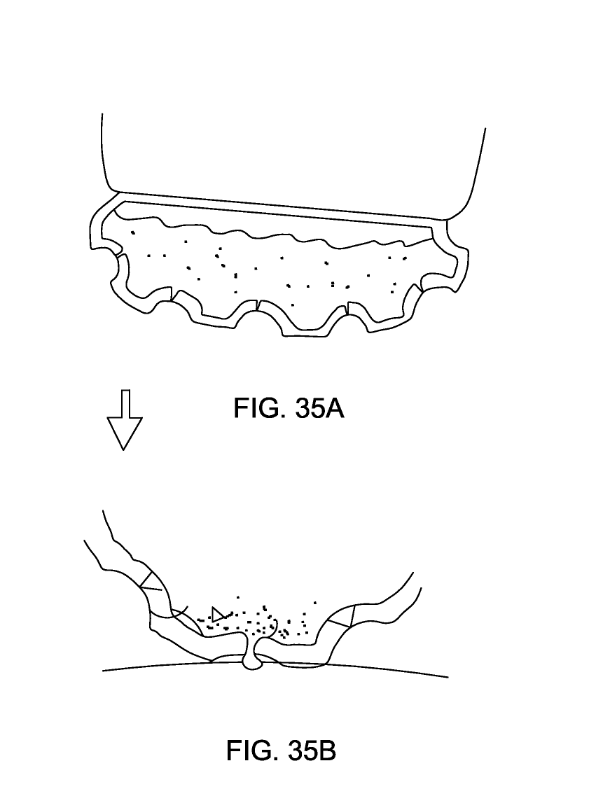

FIG. 35A is a section view of a push to dispense fluid dispenser. FIG. 35B is a section view of the dispenser of FIG. 35A illustrating how the delivery pores open when the tip is depressed.

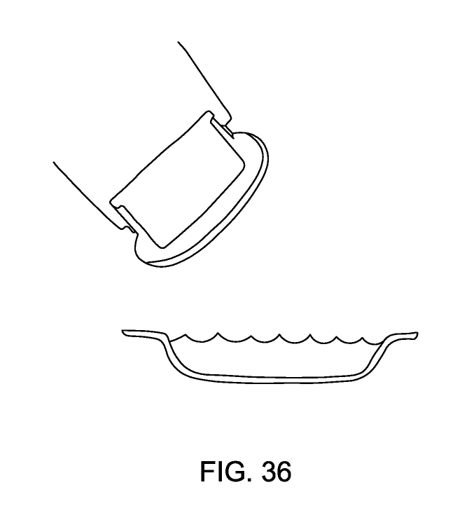

FIG. 36 is an isometric view of a disposable sleeve on an applicator and particle containing dipping tray.

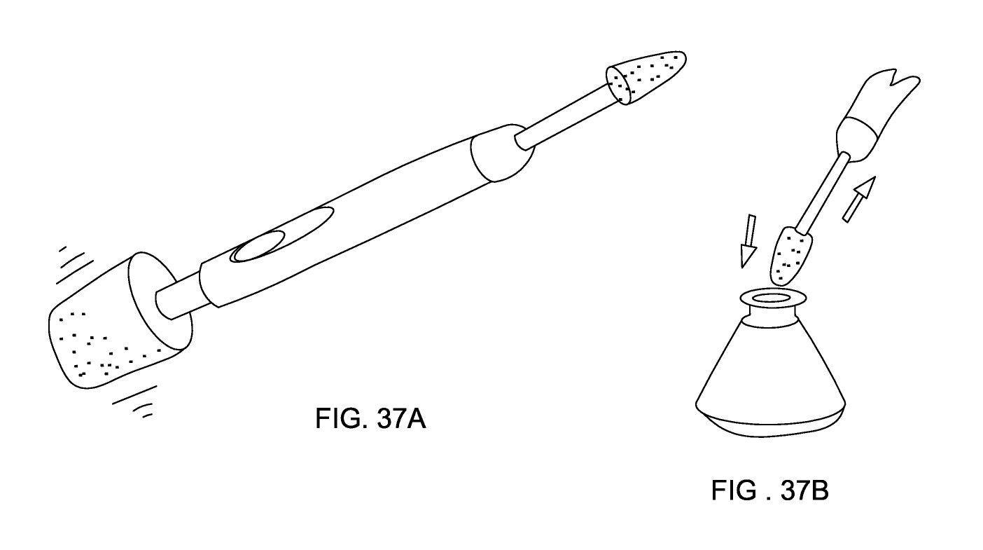

FIG. 37A is an isometric view of a double ended applicator. FIG. 37B is an enlarged view of the fluid dispenser tip of the device in FIG. 37A in use with a dip applicator.

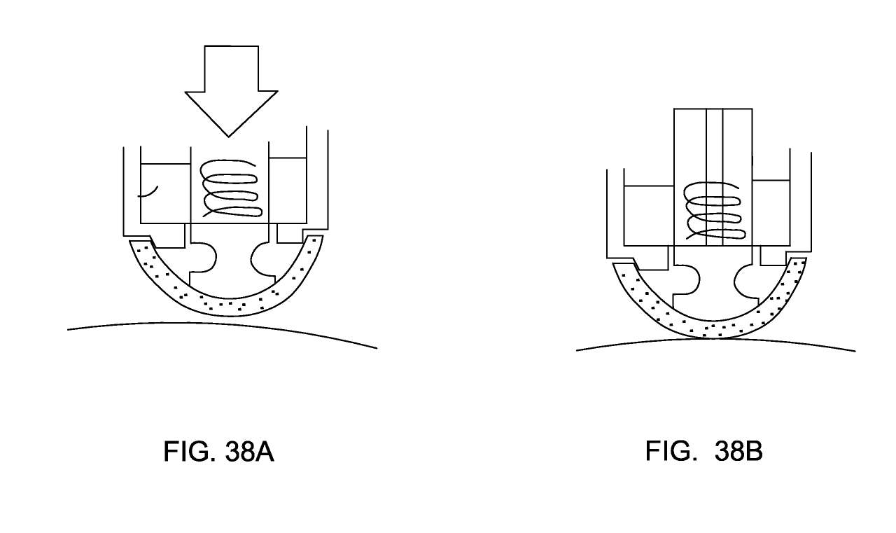

FIGS. 38A and 38B illustrate a cross section view of a push to dispense fluid delivery and applicator in the closed and fluid release configurations respectively.

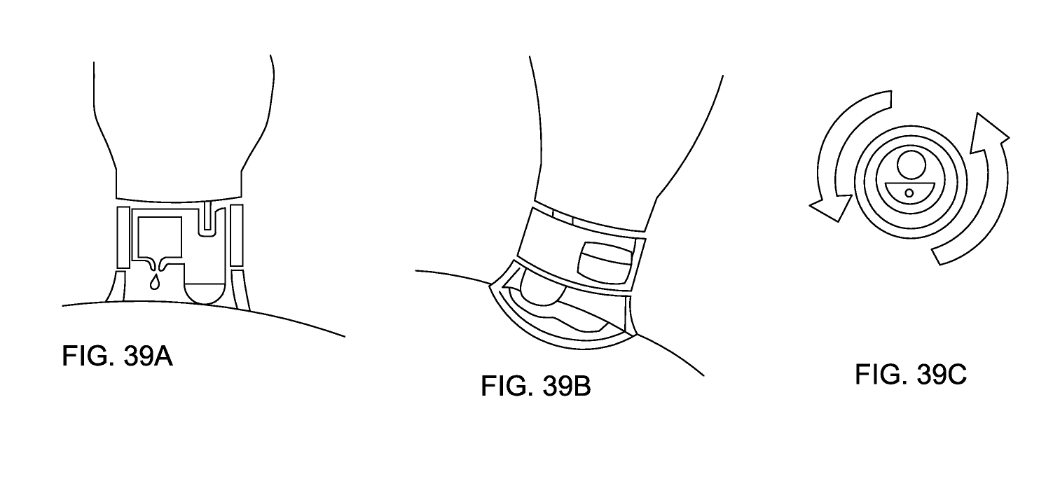

FIG. 39A is a cross section view of spinning fluid dispenser and massage roller. FIG. 39B is an isometric partial section view of the device of FIG. 39A. FIG. 39C is a top down view of the device of FIG. 39A illustrating the rotating motion.

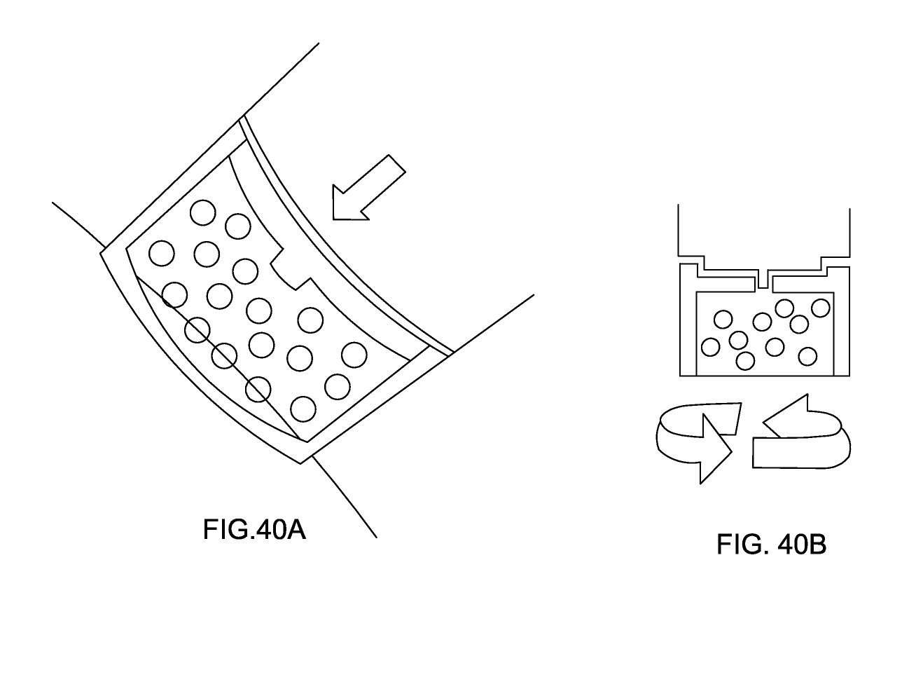

FIGS. 40A and 40B are isometric and cross section views of a fluid dispenser.

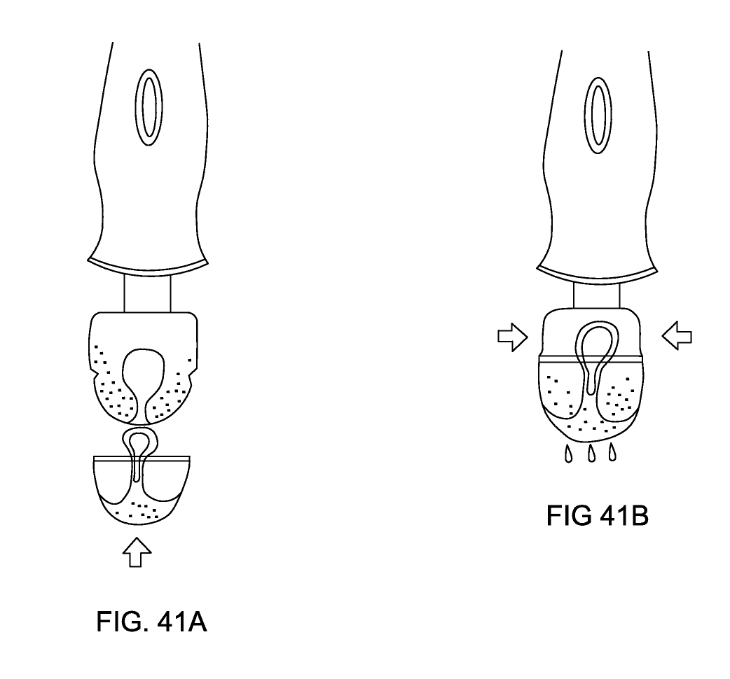

FIGS. 41A and 41B illustrate, respectively, a disposable dispensing cap before and after attachment to a mating receiver in an applicator.

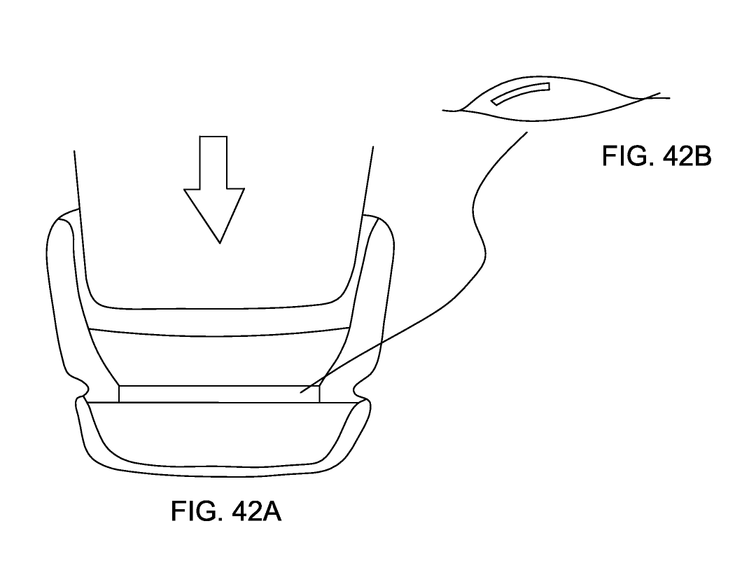

FIG. 42A illustrates a section view of an applicator and foil packet particle packet prior to use. FIG. 42B is an isometric view of the foil packet of FIG. 42A.

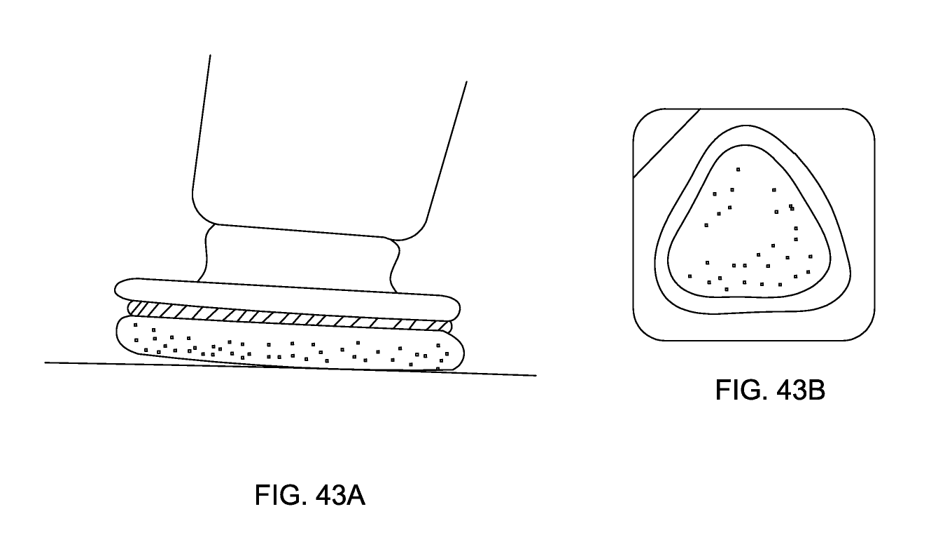

FIG. 43A illustrates a section view of an applicator and particle pad in use. FIG. 43B is a top view of the pad in FIG. 43A.

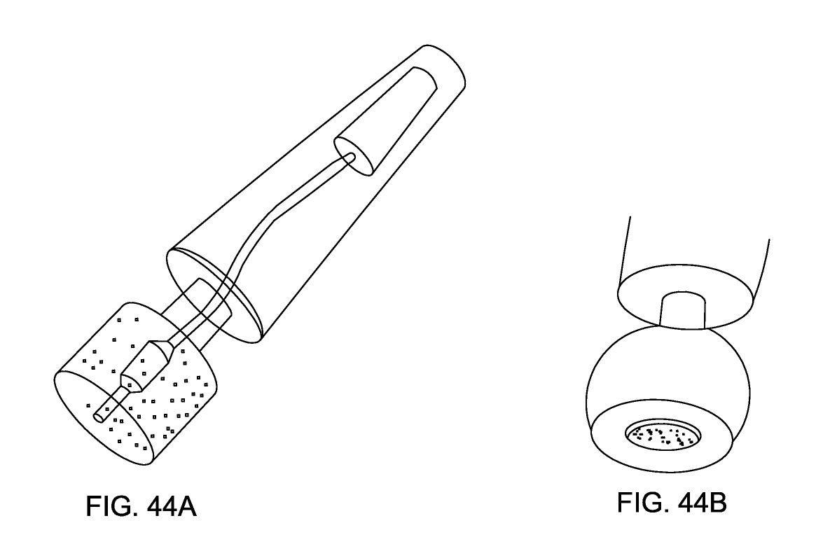

FIG. 44A is an isometric view with exposed interior components of an applicator.

FIG. 44B is an enlarged view of an alternative configuration of the distal end of the applicator in FIG. 44A.

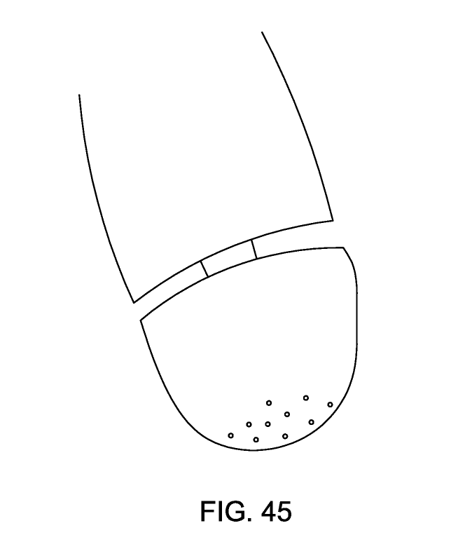

FIG. 45 is an isometric view of the distal end of an applicator having one or more elements to detect skin contact.

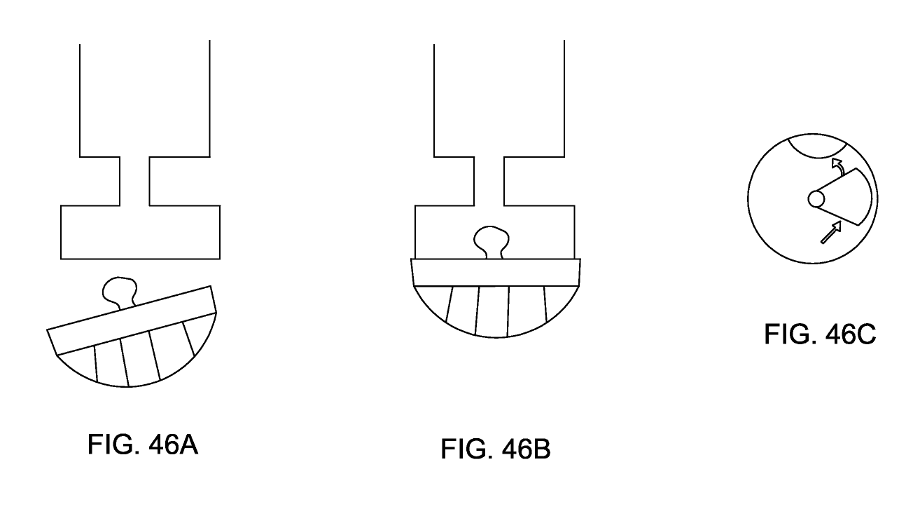

FIGS. 46A and 46B illustrate, respectively, a side view of an applicator and disposable dispenser separated and attached. FIG. 46C is a top down view of a spinning fluid dispenser that interacts with the raised ear of the disposable dispenser.

FIG. 46A is a partial section view of the device of FIG. 39A. FIG. 39C is a top down view of the device of FIG. 39A illustrating the rotating motion.

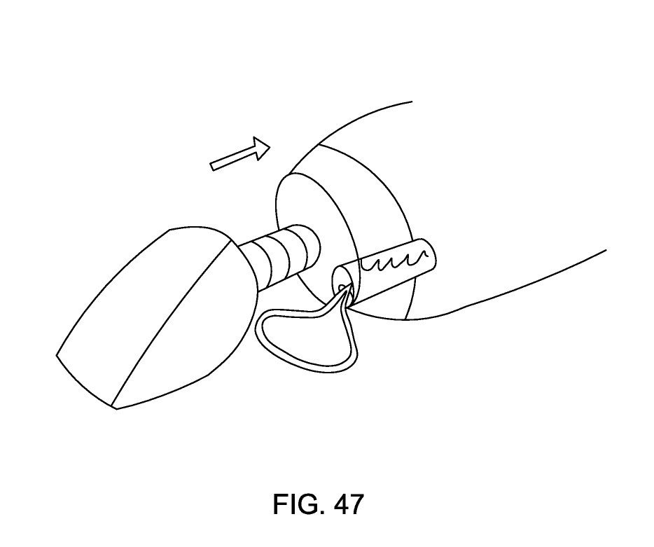

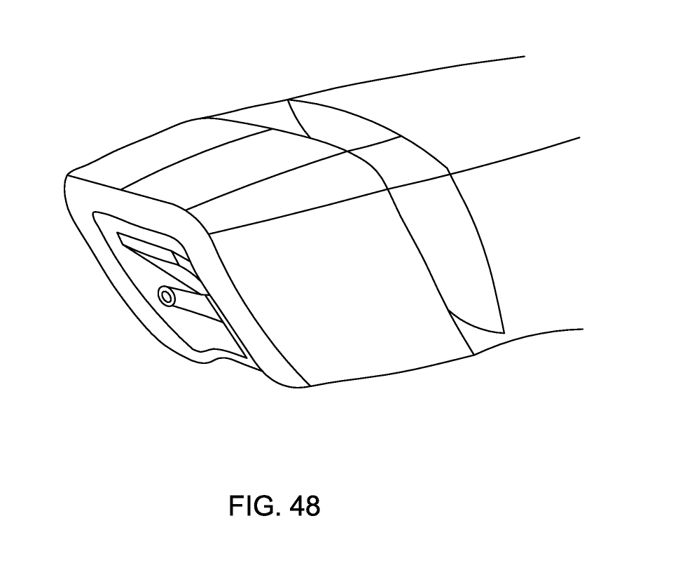

FIG. 47 is an isometric view of a dispenser having a massager head and a wicking fluid delivery loop.

FIG. 48 is an enlarged isometric view of the distal end of a vibrating delivery head having a fluid tip and air nozzle delivery system.

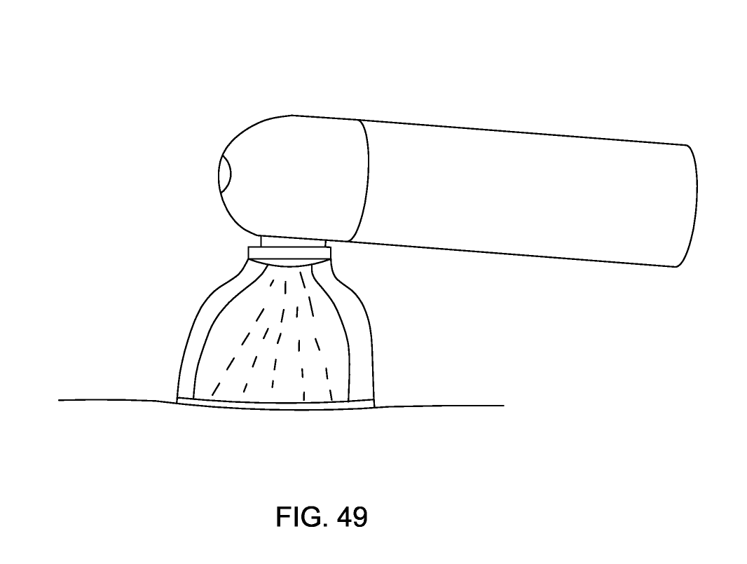

FIG. 49 is an exposed side view of a spray done fluid delivery device.

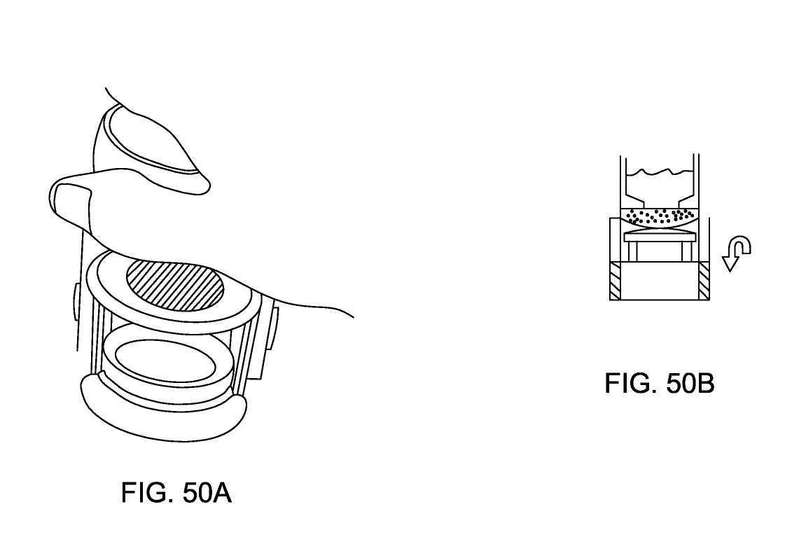

FIGS. 50A and 50B illustrate isometric and side views, respectively, a fluid dispenser.

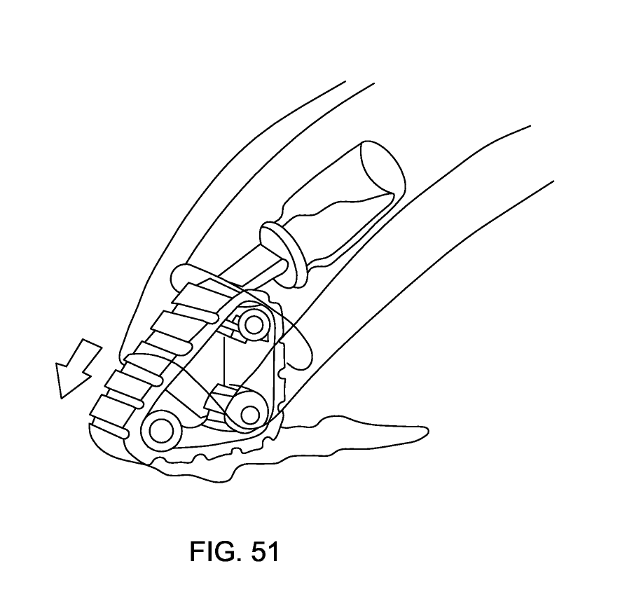

FIG. 51 is a isometric view with exposed interior components of a track head fluid dispenser.

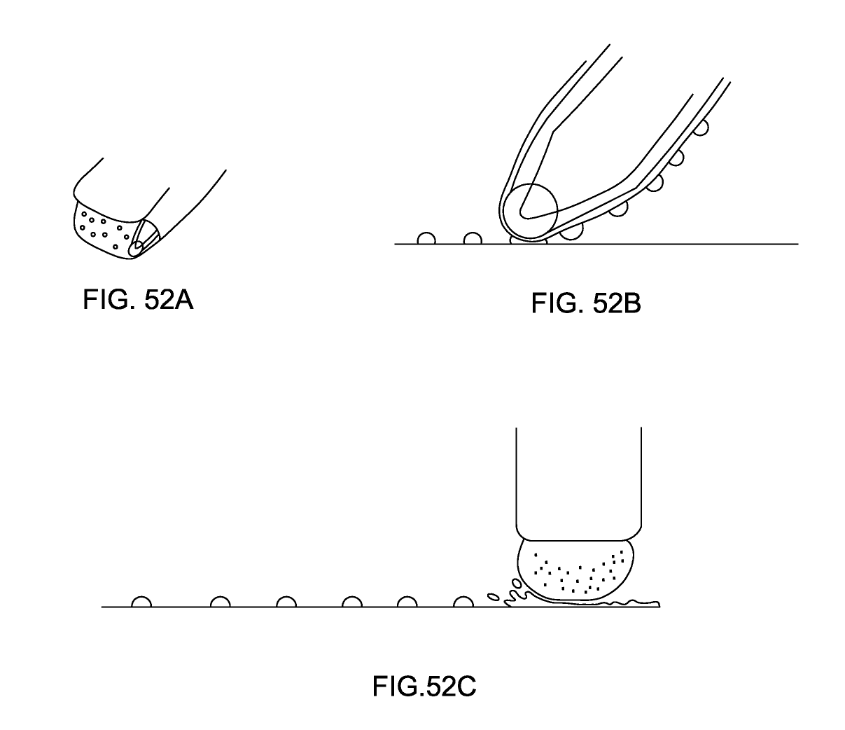

FIG. 52A illustrates a view of the distal end of a particle applicator. FIG. 52B is a side view of the delivery device of FIG. 52A applying particles to the skin. FIG. 52C is a side view of an applicator used to disperse the droplets provided in FIG. 52B.

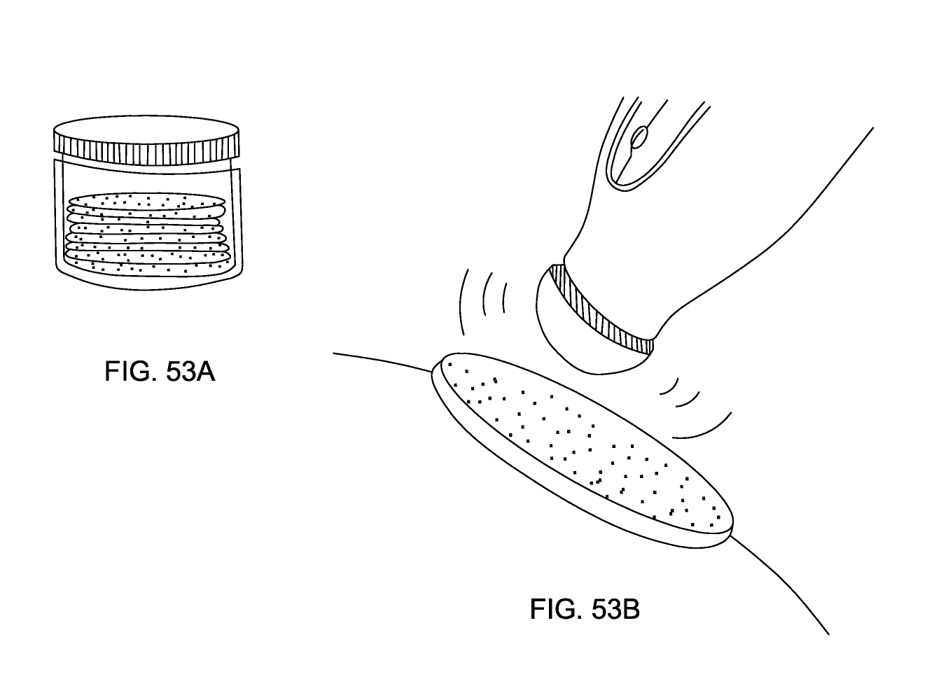

FIG. 53A illustrates a container of pads pre-moistened with a particle formulation.

FIG. 53B is an isometric view of an applicator used in conjunction with a pad from FIG. 53A to deliver particles to the skin.

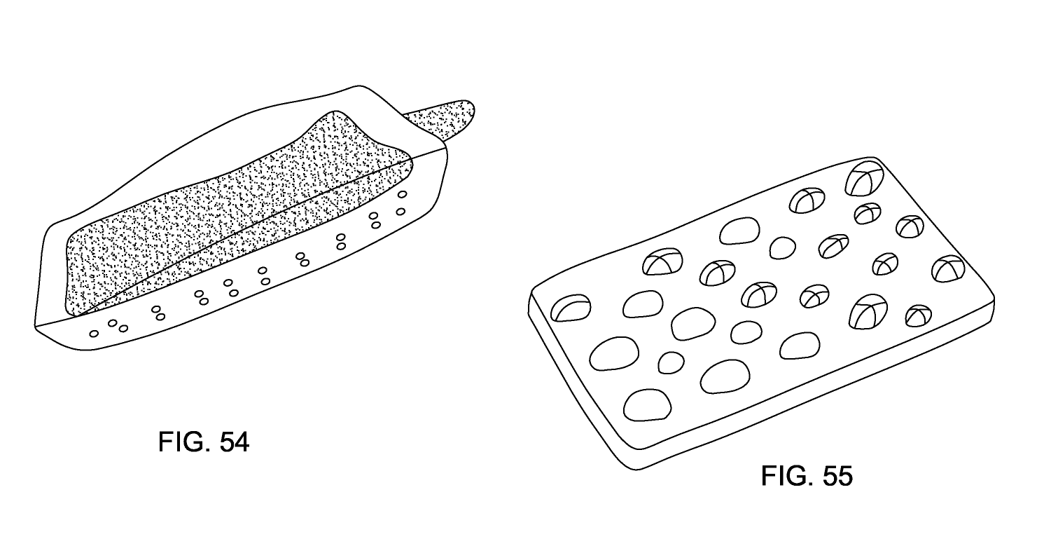

FIG. 54 is an isometric view of an adhesive particle delivery pouch.

FIG. 55 is an isometric view of an adhesive particle delivery patch having multiple individual particle formulation capsules.

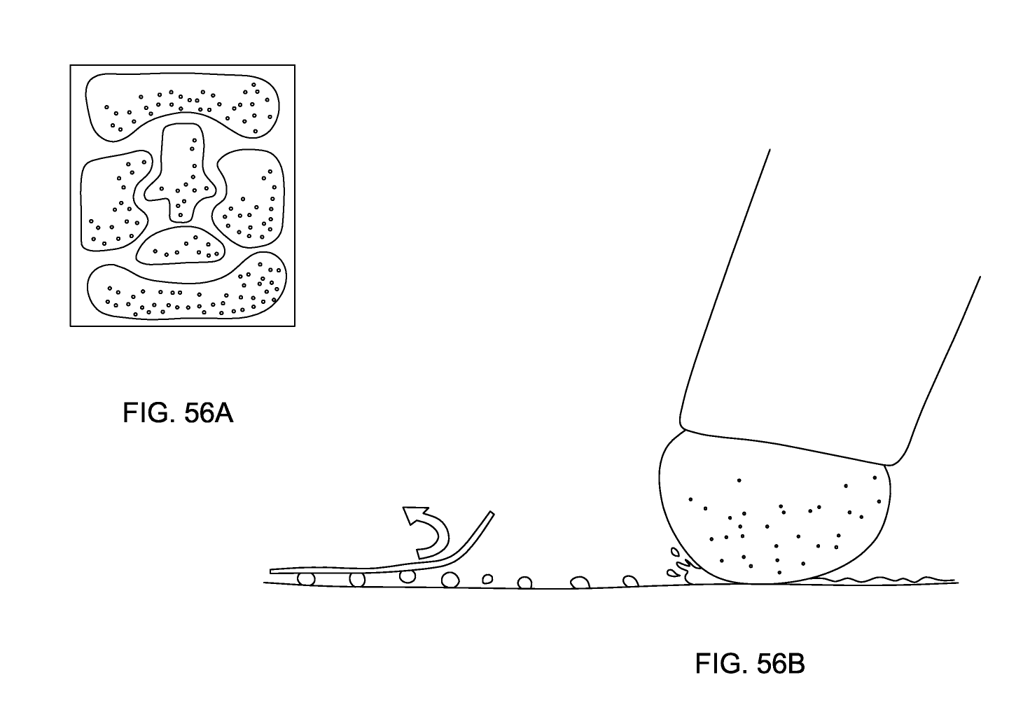

FIG. 56A is an isometric view of a set of pre-shaped adhesive particle delivery pads.

FIG. 56B illustrates the use of an applicator to deliver particles from a pad in FIG. 56A.

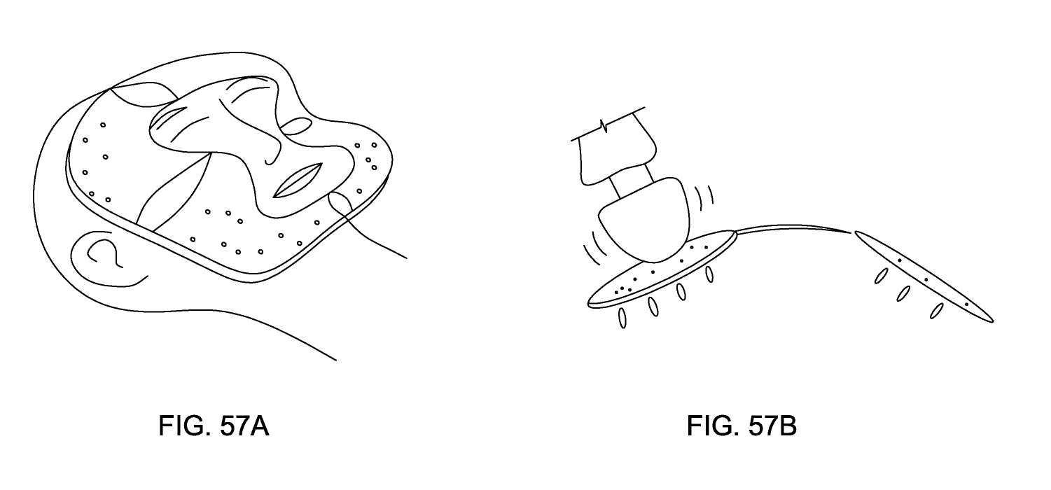

FIG. 57A is an isometric view of a set of pre-shaped adhesive particle delivery pad shaped for the perimeter of the face. FIG. 57B illustrates the use of an applicator to deliver particles from a pad in FIG. 57A.

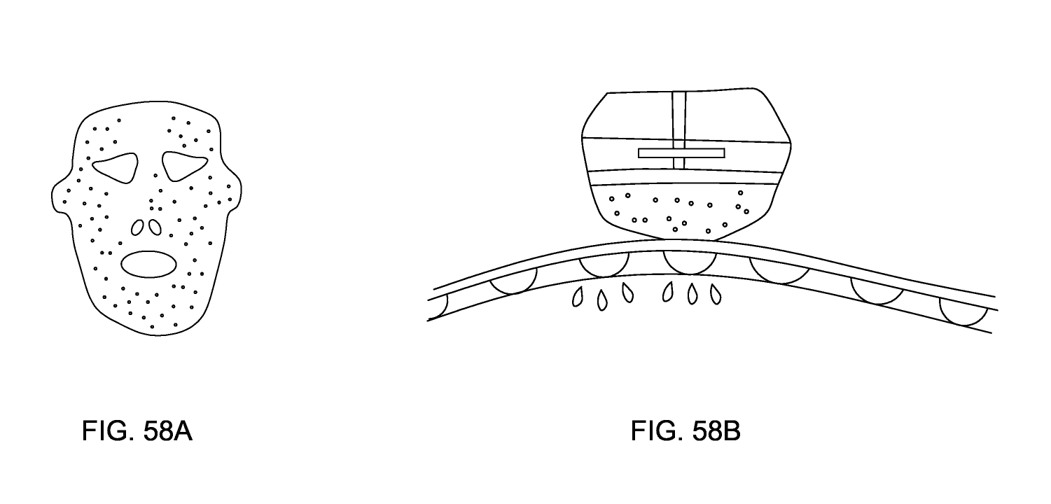

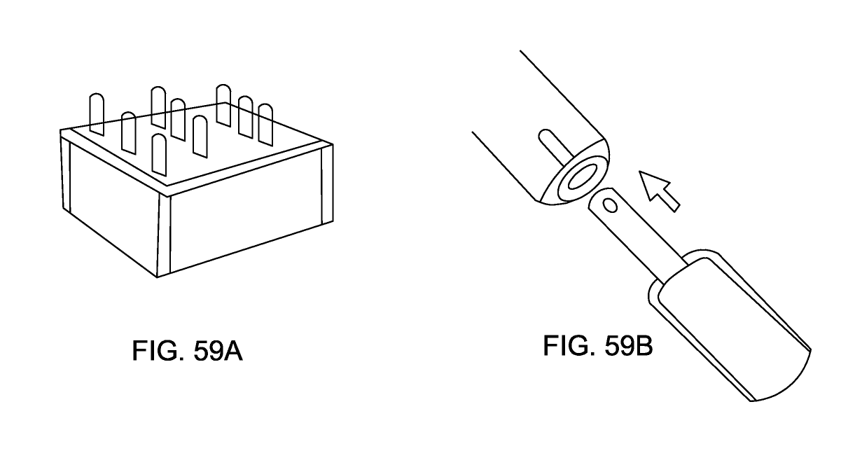

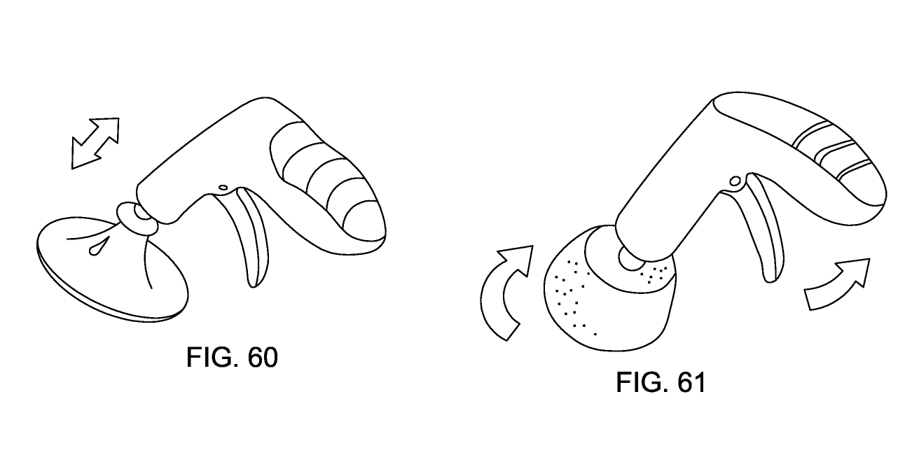

FIG. 58A is an isometric view of a set of pre-shaped adhesive particle delivery pad shaped for the face. FIG. 58B illustrates the use of an applicator to deliver particles from a pad in FIG. 58A.

FIG. 59A illustrates a plurality of frozen particle formulations. FIG. 59B illustrates one of the particle formulations of FIG. 59B prior to insertion into a suitable applicator used to liquefy the particle formation for penetration into skin target sites.

FIG. 60 is an isometric view of a suction based dispenser and applicator.

FIG. 61 is an isometric view of a trigger activated rotating massage applicator.

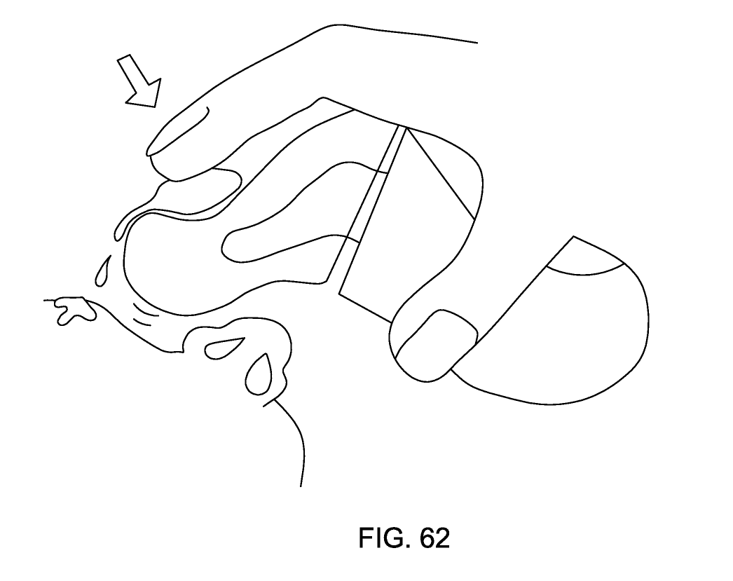

FIG. 62 is an isometric view of a palm held applicator with a fingertip activated fluid dispenser.

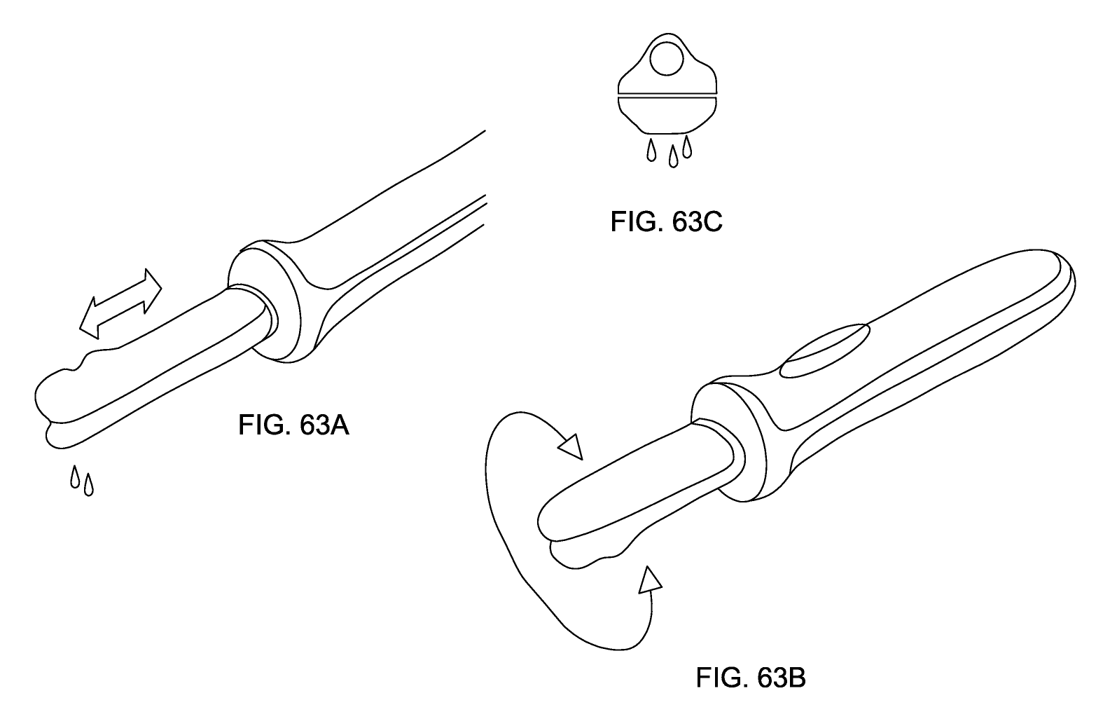

FIGS. 63A and 63B are isometric views of a dual sided dispenser and applicator with the fluid dispenser down (FIG. 63A) and the massager down (FIG. 63B). FIG. 63C is an end view of FIG. 63A.

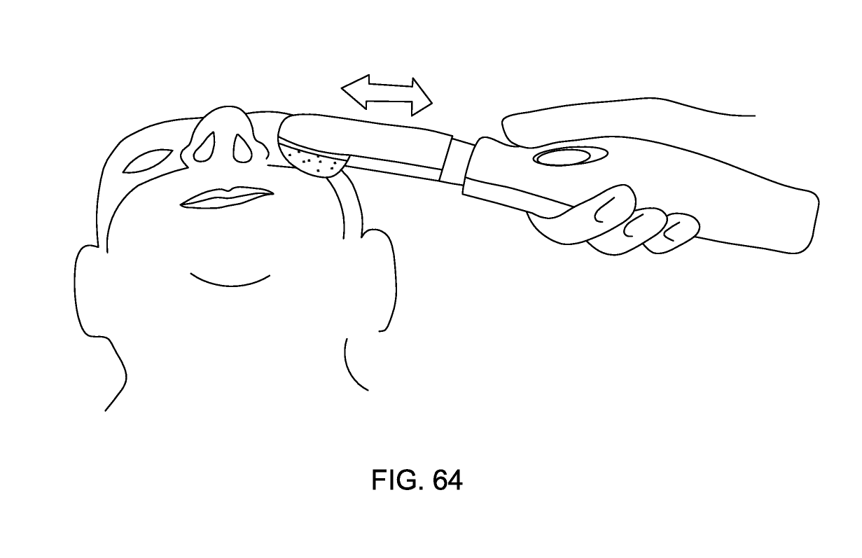

FIG. 64 is an isometric view of an alternative wand based design.

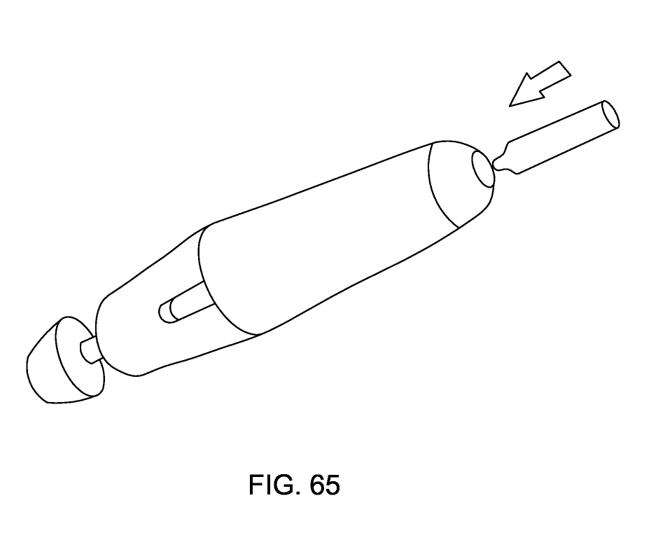

FIG. 65 is a dispenser having an air cartridge assisted delivery configuration.

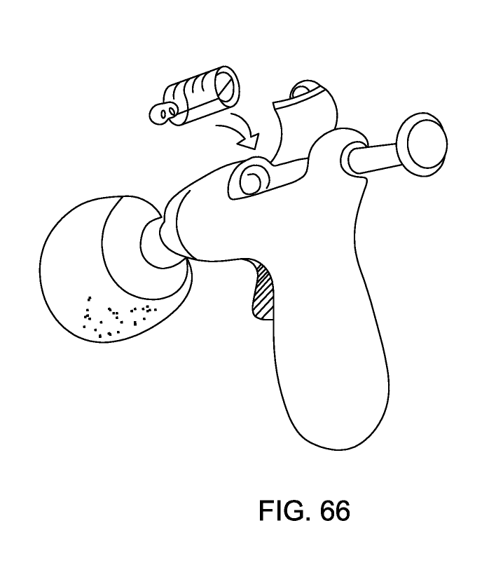

FIG. 66 is an isometric view of a trigger activated hand held applicator prior to insertion of a disposable fluid cartridge.

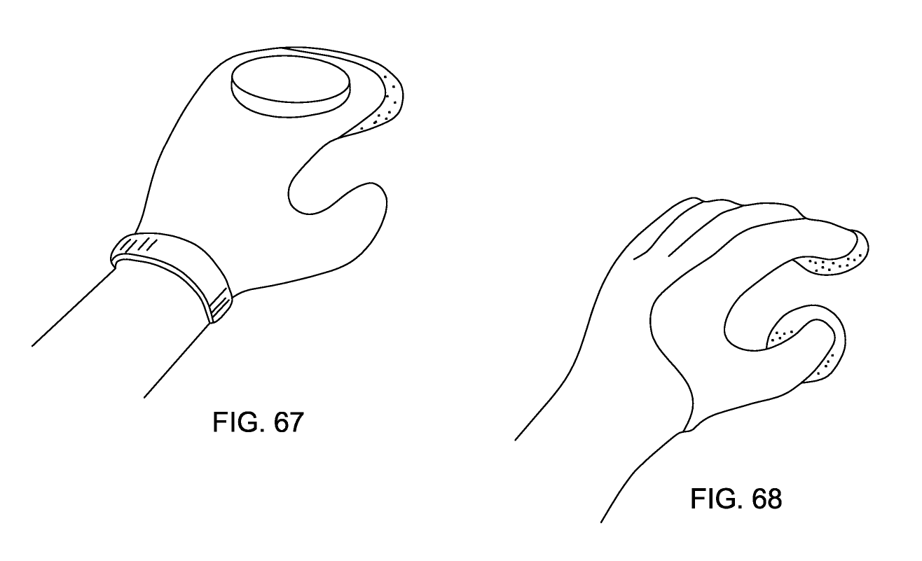

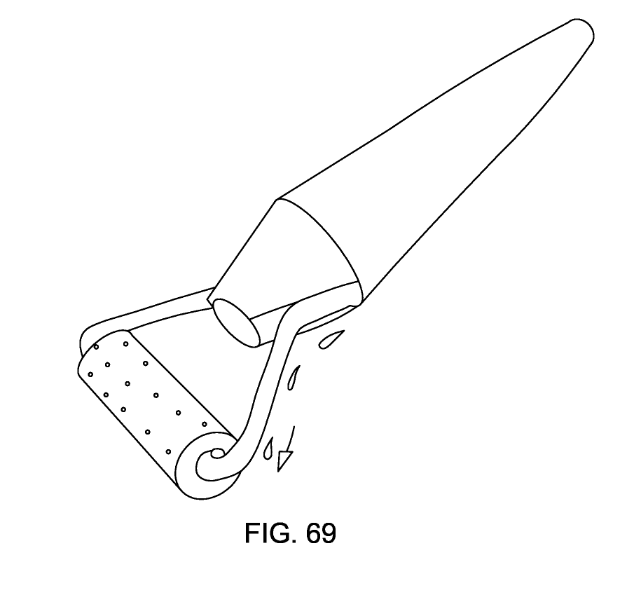

FIG. 67 is an isometric hand held massager and fluid delivery device.

FIG. 68 is an isometric view of a hand held massager and fluid delivery device that covers less of the hand than the embodiment of FIG. 67.

FIG. 69 is an isometric view of a rolling massager and fluid dispenser.

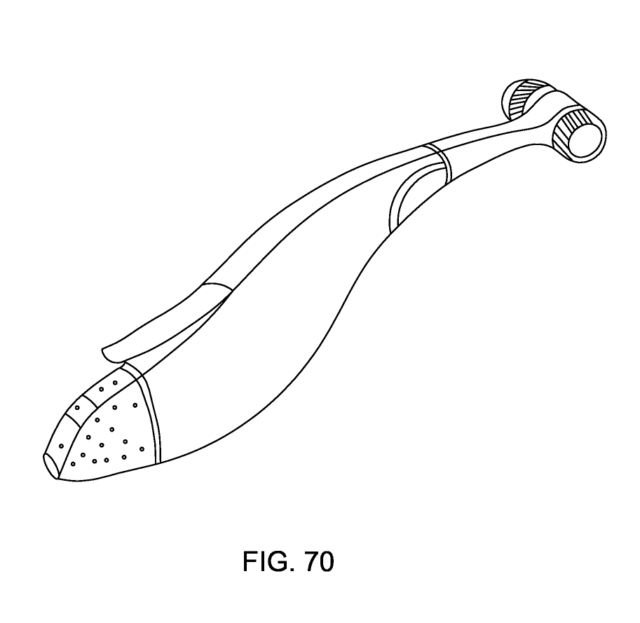

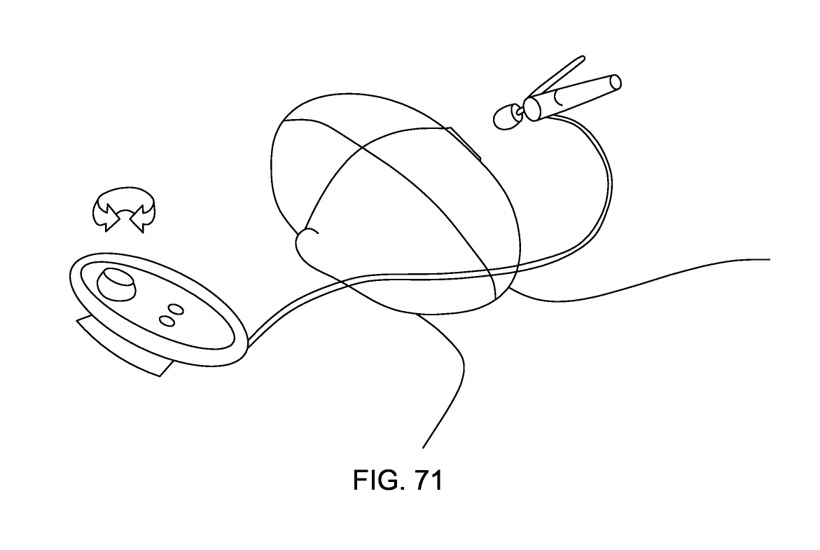

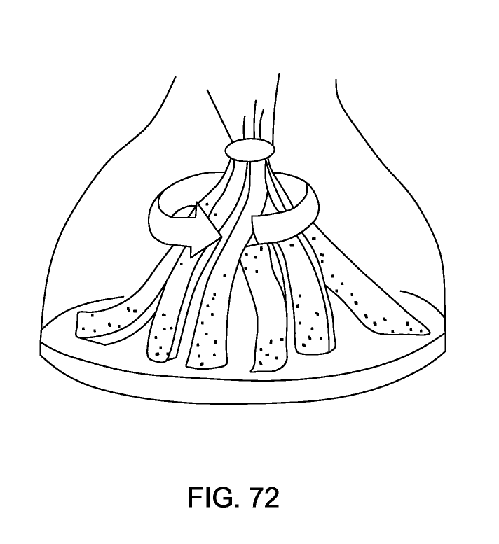

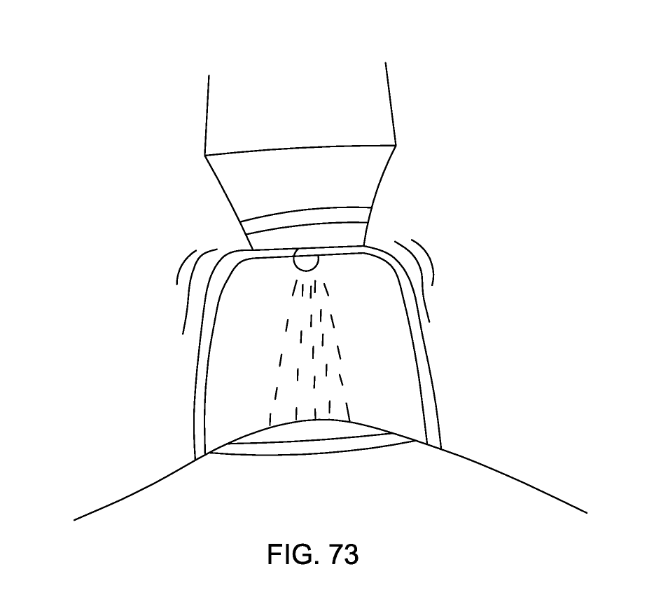

FIG. 70 is an isometric view of a dual ended dispenser.

FIG. 71 is an isometric view of another hand held applicator with separate drive and power system as illustrated above in FIGS. 14A-15B.

FIG. 72 is a side view of a spinning dispenser pad.

FIG. 73 is a side view of the distal end of an applicator using a combination of suction and vibration for particle delivery.

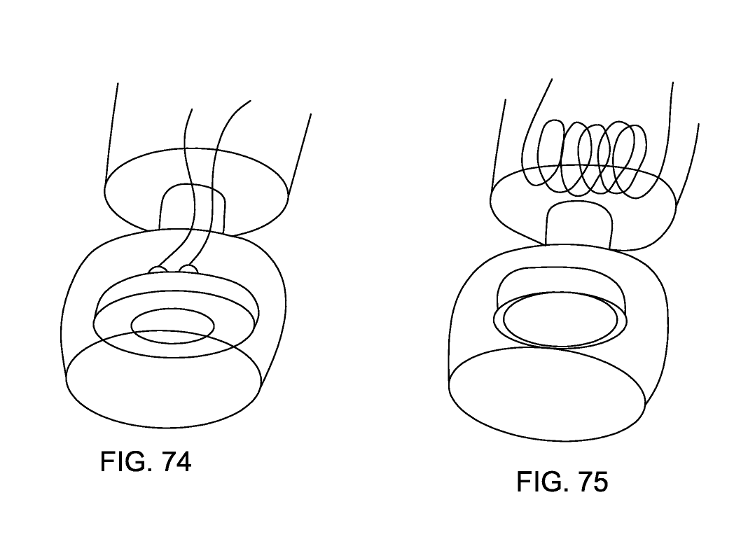

FIG. 74 is a section view of the distal end of an applicator using piezoelectric vibration for particle delivery.

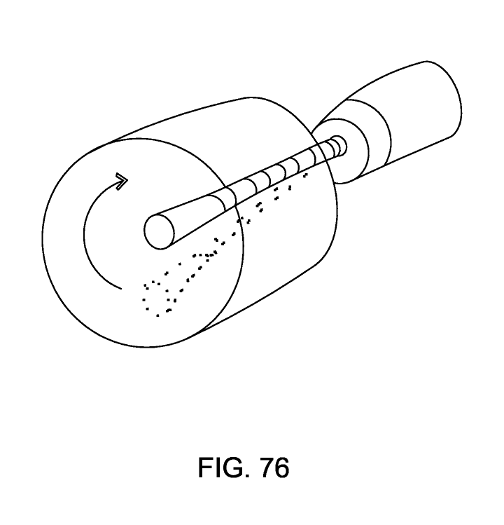

FIG. 75 is a section view of the distal end of an applicator using induction coils and a magnet for particle delivery.

FIG. 76 is a view of a distal end of an applicator having an alternative vibration device.

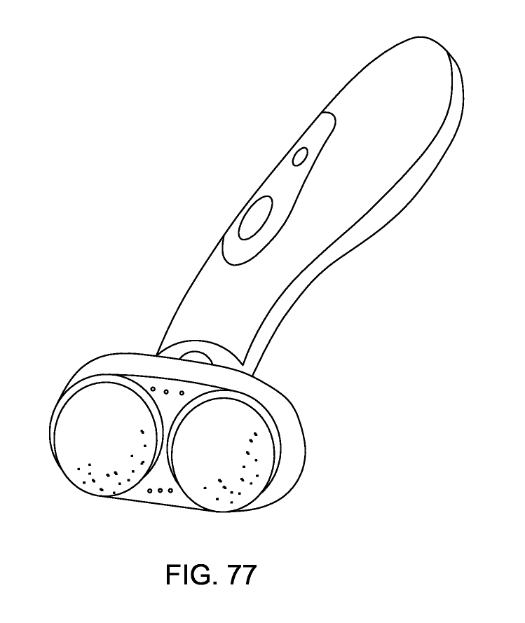

FIG. 77 is an isometric view of an applicator with two rotating heads.

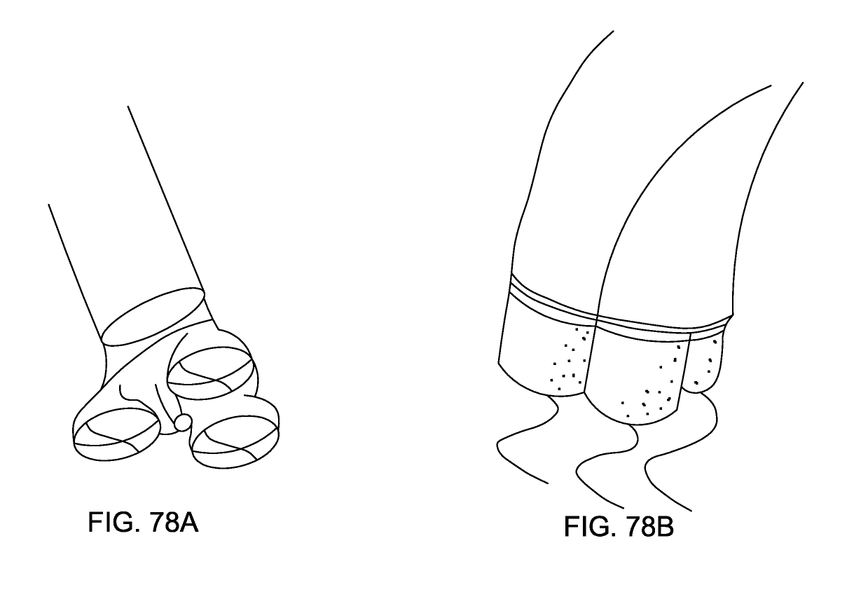

FIGS. 78A and 78B are isometric and side views of an applicator with three rotating heads.

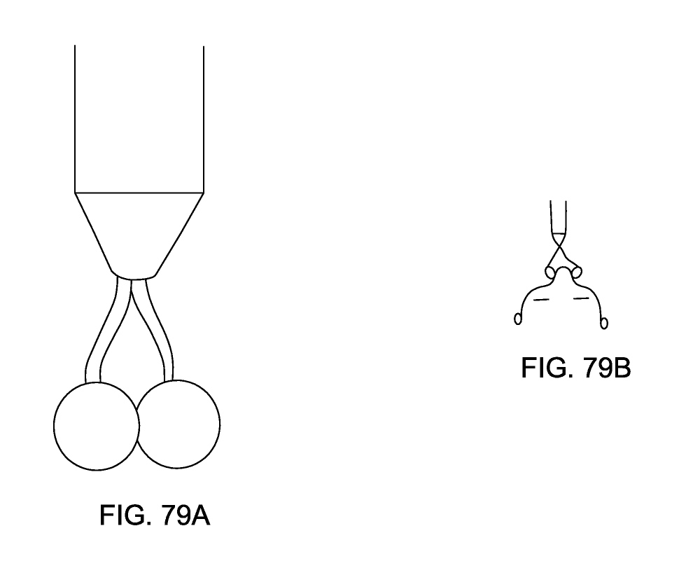

FIG. 79A illustrates a side view of an applicator having two vibrating spheres. FIG. 79B illustrates the applicator in FIG. 79A in use on a nose.

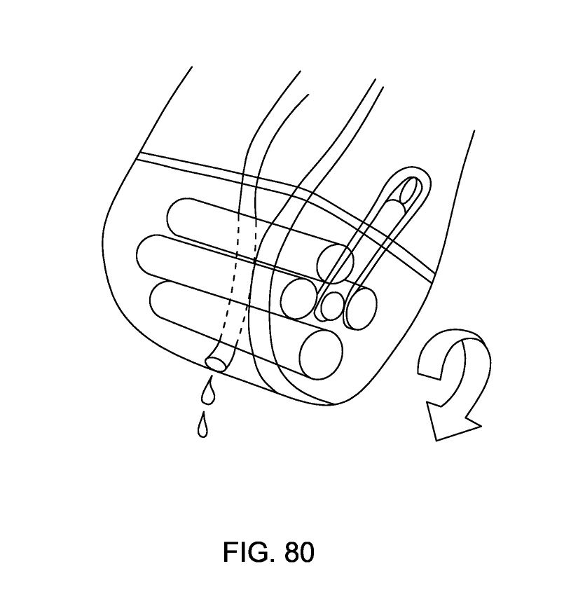

FIG. 80 illustrates an isometric and partial section view of the distal end of an applicator having a plurality of horizontal rollers.

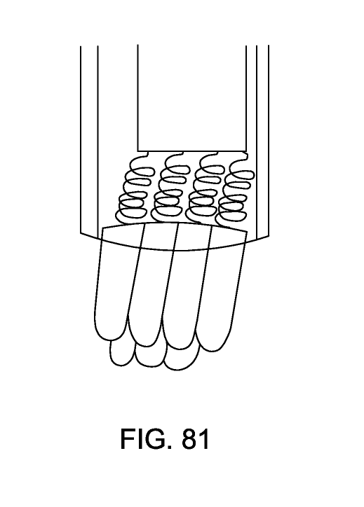

FIG. 81 is a section view of the distal end of an applicator having a plurality of individually spring loaded fingers.

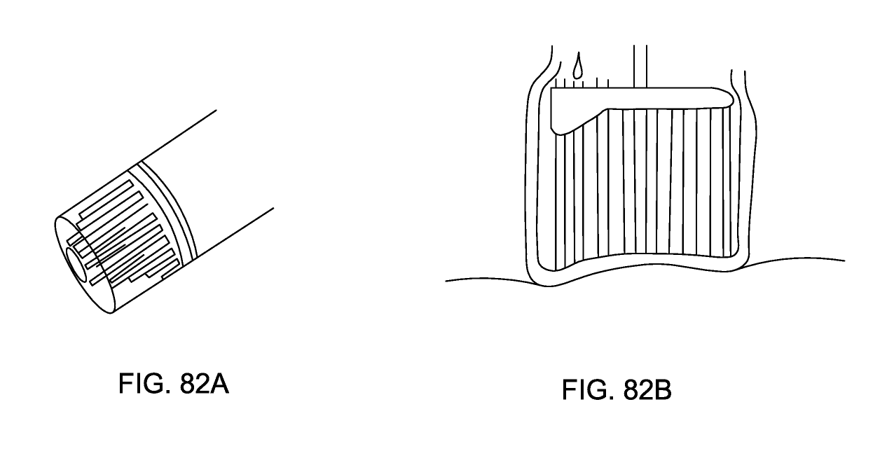

FIGS. 82A and 82B are, respectively, isometric and side views of applicator having individual pins that move via interaction with a rotating loaded ramp.

The examples and illustrations included herein show, by way of illustration and not of limitation, specific embodiments in which the storage, delivery or facilitation of particle application to the skin or target site may be practiced. While certain specific embodiments are illustrated in use together in the above illustrative examples, the various aspects of the invention are not so limited. Various alternative configurations are possible by combining one or more design aspects or components described above into an additional embodiment or specific configuration. By way of example, an applicator head as illustrated or described in one of FIG. 1F, 2B, 3C, 4B-4F, 5B, 6A, 7A, 8A, 9, 10A, 11A-11D, 12A or 13A may be adapted and configured for use with one or a combination of different motion inducing devices or fluid delivery modes as illustrated in, for example, FIGS. 32-35B, 38A-39B, 41A, 41B, 43A-44B, 46A-51 and 72-82. In still further aspects, any of the illustrated or described particle or material containers or housings may be adapted and configured by shape, size or addition of one or more mating or keying features for use in any of the various delivery devices or modes described herein. The mating or keying features are not limited to the particle container exterior surfaces but could also be on one or both of the distal ends. In much the same way, the applicator receiver for mating to the particle container may also be specifically keyed to a particular formulation within a specific container. In this way a user may also be guided to ensure that the proper material container is being inserted into a suitable applicator or is being properly used based on the procedure(s) for which the material is being applied or the applicator is being used. The mating or keying features provided at the container—applicator receiver interface include, by way of example and not limitation, tab-slot, pin-notch, mating splines, knob-loop, ratchets, male-female connectors, friction fittings, and other suitable two-, or more, part joints that will secure a specific particle container into the container receiver socket of an applicator.

In one aspect, the operation of a delivery device for the delivery of a delivery fluid is the desired therapy. In this case, the operation of the delivery device is a complete treatment operation. In another aspect, the operation of the delivery device, applicator or particle formulation assistance for the delivery of a delivery fluid precedes or follows another treatment or another desired therapy. In this case, the operation and use of the delivery device is one part of a multi-part therapy. In one specific example of a multiple part therapy is the use of the delivery system to deliver a fluid, a formulation of particles, shells, pharmaceuticals, liposomes, other treatment agents or pharmacologic materials onto, into or within a structure within a treatment or delivery site followed by a further treatment of the delivery or treatment site. In one specific example the further treatment is providing an activating energy to a fluid, a formulation or a pharmacologic material. Exemplary fluids, formulations and treatments are described in U.S. Pat. No. 6,183,773; U.S. Pat. No. 6,530,944; U.S. Published Patent Application US 2013/0315999 and U.S. Published Patent Application US 2012/0059307, each of which is incorporated herein in its entirety. Additionally or optionally, one or more of the delivery device operating parameters, device configuration, and/or methods of use of a delivery system described herein may be modified based upon one or more characteristics of the location of the treatment site on the body, the use of an integrated or separate particle formulation container, the type or amount of delivery fluid, a component of the delivery fluid or a particle within the delivery fluid being used in a specific configuration.

Though the description is for an apparatus for delivery of a formulation generally and in some specific examples formulations comprising particles, the invention is not so limited. In addition to formulations, including formulations with particles, the various devices, methods and systems described herein also include their use as adapted and configured for delivery of dissolved materials. Dissolved materials may take on a wide variety of forms such as light absorbing dyes and colors, drugs, and pro-drugs including PDT drugs. Additionally or alternatively, the delivery of dissolved materials or particles may also include the use of a formulation for the delivery to the skin that changes state from storage to delivery. In one specific embodiment, a formulation (i.e., particle comprising or dissolved material comprising formulation) as described herein is combined with coconut oil to form a paste (at room temperature) to aid in application to a treatment site. Thereafter, the coconut oil based particle formulation is liquefied upon application of massage or other mechanical delivery.

Although various illustrative embodiments are described above, any of a number of changes may be made to various embodiments without departing from the scope of the invention as described by the claims. For example, the order in which various described method steps are performed may often be changed in alternative embodiments, and in other alternative embodiments one or more method steps may be skipped altogether. Optional features of various device and system embodiments may be included in some embodiments and not in others. Therefore, the foregoing description is provided primarily for exemplary purposes and should not be interpreted to limit the scope of the invention as it is set forth in the claims.

The examples and illustrations included herein show, by way of illustration and not of limitation, specific embodiments in which the subject matter may be practiced. As mentioned, other embodiments may be utilized and derived there from, such that structural and logical substitutions and changes may be made without departing from the scope of this disclosure. Such embodiments of the inventive subject matter may be referred to herein individually or collectively by the term “invention” merely for convenience and without intending to voluntarily limit the scope of this application to any single invention or inventive concept, if more than one is, in fact, disclosed. Thus, although specific embodiments have been illustrated and described herein, any arrangement calculated to achieve the same purpose may be substituted for the specific embodiments shown. This disclosure is intended to cover any and all adaptations or variations of various embodiments. Combinations of the above embodiments, and other embodiments not specifically described herein, will be apparent to those of skill in the art upon reviewing the above description.

1-4. (canceled)

5. A device for delivering a fluid to skin while massaging said skin comprising:
A device body, said device body comprising a battery and a battery operated motor in electrical communication with said battery;
A massage head coupled to said motor in a manner effective to massage said skin;
Said device body further comprising a receptacle for securely holding a delivery container;
Said receptacle having a piston which is advanced in small increments within said delivery container in response to an input signal supplied by depressing a button on a surface of said device body, said piston advances dispensing a small volume of fluid from said delivery container to said skin adjacent to where said massage head is massaging said skin.

6. The device of claim 5 wherein said delivered volume per increments between 0.1 and 0.3 ml.

7. A disposable delivery element comprising:
A container having a sterile interior comprising a volume of between about 0.5 and 10 ml;
Said container further comprising a delivery member having a sealed state and a delivery state through which a composition comprising particles can be delivered; and
Said container further comprising a member effective to pressurize said fluid within said container so as to deliver said composition through said delivery member in said delivery state.

8. The disposable delivery element of claim 7 further comprising a shape adapted for insertion into a delivery device.
